(19) United States
(12) Patent Application Publication
(10) Pub. No.: US 2024/0269382 A1
(43) Pub. Date: Aug. 15, 2024

(54) CLOSED-LOOP HOME DIURETIC THERAPY SYSTEM

(71) Applicant: TERUMO KABUSHIKI KAISHA, Tokyo (JP)

(72) Inventors: Shuji UEMURA, San Francisco, CA (US); Matthew John ZWEBER, Mountain View, CA (US); Koray SAHIN, Mountain View, CA (US); Sean Michael ROTHENBERGER, Pennsburg, PA (US)

(21) Appl. No.: 18/567,739

(22) PCT Filed: Jun. 6, 2022
(86) PCT No.: PCT/IB2022/055223

§ 371 Date: Dec. 6, 2023

Related U.S. Application Data
(60) Provisional application No. 63/209,372, filed on Jun. 10, 2021.
(60) Provisional application No. 63/294,340, filed on Dec. 28, 2021.

(51) Int. Cl. A61M 5/172

(52) U.S. Cl. CPC A61M 5/1723; A61M 2005/1726; A61M 2205/18; A61M 2205/3334; A61M 2205/3375; A61M 2205/3379; A61M 2205/3553; A61M 2209/088; A61M 2230/20; A61M 2230/30

(57) ABSTRACT

A closed-loop home diuretic therapy system includes a monitoring device attached to the patient, the device having a sensor to detect a wall of a bladder, an accelerometer to detect an orientation of device to account for bladder positions, and a processor to determine an amount of urine in the bladder based on the detected wall and the orientation and calculate urine output volume based on the determined amount of urine, and a diuretic administration device configured to communicate with the monitoring device, the In administration device having a subcutaneous injection site, a diuretic delivery and storage device to deliver diuretic stored therein to the injection site, and a control circuit configured to regulate the volume and rate of diuretic delivered from the storage device to the injection site based on the calculated urine output volume and a target volume and rate of diuretic.

### CROSS REFERENCE TO RELATED APPLICATIONS

The present application claims priority to U.S. Provisional Patent Applications No. 63/209,372 filed Jun. 10, 2021 and No. 63/294,340 filed Dec. 28, 2021, which are herein incorporated by reference in their entirety.

### TECHNICAL FIELD

Embodiments of the present disclosure generally relate to a system consisting of medical devices, more particularly a closed-loop system and methods for achieving home diuretic therapy for people with heart failure (HF). The system and methods employ a urine output volume monitoring device, a diuretic administration device, and a closed-loop algorithm to regulate the diuretic dose based on the urine output volume, which are configured, for example, to achieve fluid balance in people who suffer from fluid volume overload caused by HF.

### BACKGROUND

Diuretic administration is one of the most commonly used therapies for heart failure (HF) patients to treat fluid volume overload and achieve a state of euvolemia. This state of euvolemia is usually maintained by taking oral diuretics at home. However, oral diuretics may encounter ineffectiveness due to diuretic resistance which substantially diminishes the effect of diuretic. Noncompliance or nonadherence by the patient to the prescribed regimen may also contribute to the ineffectiveness of oral diuretics.

Currently these issues are addressed by increasing the dose of the diuretic, having HF patients go to hospitals or clinics to receive intravenous (IV) diuretic therapy, or both. However, increasing the dose of the diuretic is not always effective particularly for oral diuretics, and receiving IV diuretic therapy is long, cumbersome, and not cost effective for both providers and payers.

It would be desirable for HF patients to receive diuretic therapy to maintain euvolemia at home, which can reduce the concerns of diuretic resistance, noncompliance or nonadherence, or unnecessary hospital or clinic visits to receive IV diuretic therapies. This may be achieved by a closed-loop diuretic therapy system in which the diuretic dose is regulated based on the corresponding urine output volume.

U.S. Pat. Nos. 9,655,520 and 10,537,281 disclose a closed-loop system for decongestive therapy titration for HF patients using data collected from sensors. The system, however, relies on implantable sensors that are difficult to deploy in a home setting. In addition, the system requires the participation of a physician, caregiver, and/or clinician in the closed-loop to adjust or titrate the decongestive therapy, because the evaluation of the effectiveness of the decongestive therapy requires a skilled practitioner's interpretation of the physiological parameter or condition of the patient measured by the sensors.

### SUMMARY

One or more embodiments provide a closed-loop home diuretic therapy system that accurately determines a target diuretic dose (e.g., a target volume and rate of diuretic) to be administered to a patient based on a measured parameter. In the embodiments, urine output volume is selected as the measured parameter because it is a good measure of diuretic efficacy. Measuring the urine output volume is advantageous because it can be performed in a non-invasive way and is also simpler relative to some of the physiological parameters measured in the related art, some of which require a blood sample and/or analysis of constituents. In addition, the embodiments improve the accuracy of measuring urine output volume by accounting for different bladder positions of the patient.

A closed-loop home diuretic therapy system according to one embodiment is configured to achieve decongestion without residual volume overload in a patient. The therapy system comprises a urine output volume monitoring device to be attached to the patient, the urine output volume monitoring device having a sensor to detect a wall of a bladder of the patient, an accelerometer to detect an orientation of urine output volume monitoring device, and a processor to determine an amount of urine in the bladder based on the detected wall and the detected orientation and calculate urine output volume based on the determined amount of urine in the bladder. The therapy system further comprises a diuretic administration device configured to communicate with the urine output volume monitoring device, the diuretic administration device having a subcutaneous injection site with an injection feature for subcutaneous diuretic injection, a diuretic delivery and storage device with a pump to deliver diuretic stored therein to the subcutaneous injection site, and a control circuit having a processor that is configured to regulate the volume and rate of diuretic delivered from the diuretic delivery and storage device to the subcutaneous injection site based on the calculated urine output volume and a target volume and rate of diuretic to be administered.

### BRIEF DESCRIPTION OF THE DRAWINGS

FIG. 1 is an illustration of a closed-loop home diuretic therapy system including a urine output volume monitoring device, a diuretic administration device, and a mobile device communicating with each other by wireless technology in accordance with an exemplary embodiment.

FIGS. 2A and 2B are perspective views of a monitor housing for the urine output volume monitoring device in accordance with an exemplary embodiment.

FIGS. 2C and 2D are perspective views of a monitor housing holder in accordance with an exemplary embodiment.

FIGS. 3A through 3D are perspective views of the urine output volume monitoring device with adhesive attachments in accordance with an exemplary embodiment.

FIGS. 4A through 4D are schematics depicting different shapes and configurations of the monitor housing and the adhesive attachments in accordance with an exemplary embodiment.

FIGS. 5A through 5F are perspective views of an ultrasound sensor for the urine output volume monitoring device configured with multiple sensor elements in accordance with an exemplary embodiment.

FIG. 6A is a perspective view of an individual ultrasound sensor element configured with an angle-adjusting feature in accordance with an exemplary embodiment.

FIG. 6B is a schematic of ultrasound sensor elements for performing phased array scanning.

FIGS. 6C and 6D are schematics of ultrasound sensor elements that can be mechanically rotated as a unit.

FIG. 7 is a perspective view of the urine output volume monitoring device configured to be placed near the perineum in accordance with an exemplary embodiment.

FIG. 8 is another perspective view of the urine output volume monitoring device configured with an adjustable belt strap in accordance with an exemplary embodiment.

FIGS. 9A through 9F are schematic illustrations of the urine output volume monitoring device configured to be worn as an undergarment in accordance with an exemplary embodiment.

FIGS. 10A and 10B are perspective views of a subcutaneous injection site with attachments and array of microneedles for the diuretic administration device in accordance with an exemplary embodiment.

FIG. 10C is a perspective view of a subcutaneous injection site with attachments and a cannula for the diuretic administration device in accordance with an exemplary embodiment.

FIGS. 11A and 11B are perspective views of a drug delivery and storage unit for the diuretic administration device in accordance with an exemplary embodiment.

FIGS. 12A through 12C are perspective views of a pumping unit and a replaceable cartridge of the drug delivery and storage unit for the diuretic administration device in accordance with an exemplary embodiment.

FIG. 13 is a flowchart describing how the closed-loop home diuretic therapy system regulates the diuretic dose based on the urine output volume in accordance with an exemplary embodiment.

FIG. 14 is a flowchart describing how the urine output volume monitoring device measures and calculates the overall urine output volume from the urine measurements in the bladder in accordance with an exemplary embodiment.

FIG. 15 is a flowchart describing how the urine output volume monitoring device detects the orientation change of the device to account for different bladder positions in taking the urine measurements in accordance with an exemplary embodiment.

FIG. 16 is a flowchart describing how the urine output volume monitoring device detects the orientation change of the device to notify the user in taking the urine measurements in accordance with an exemplary embodiment.

FIG. 17 depicts measured bladder volumes and error levels thereof.

FIG. 18 depicts the coordinate system used to determine an error level of bladder volume based on accelerometer and gyroscope measurements.

FIG. 19 is a flowchart describing how the urine flow in the urethra detected by an acoustic sensor is converted to urine output volume in accordance with an exemplary embodiment.

FIG. 20 is a schematic depicting how the information and data flow in the closed-loop home diuretic therapy system in accordance with an exemplary embodiment.

FIG. 21 is a plot of urine volume versus time that is used to describe the algorithm to regulate diuretic dose.

FIGS. 22A through 22C depict data flow in the closed-loop home diuretic therapy system in accordance with an exemplary embodiment.

FIG. 23 is a flowchart of steps performed by the closed-loop home diuretic therapy system in accordance with an exemplary embodiment.

### DETAILED DESCRIPTION

Hereinafter, embodiments of the present disclosure will be described with reference to the drawings. In order to facilitate description, dimensional ratios in the drawings are exaggerated, and thus are different from actual ratios in some cases.

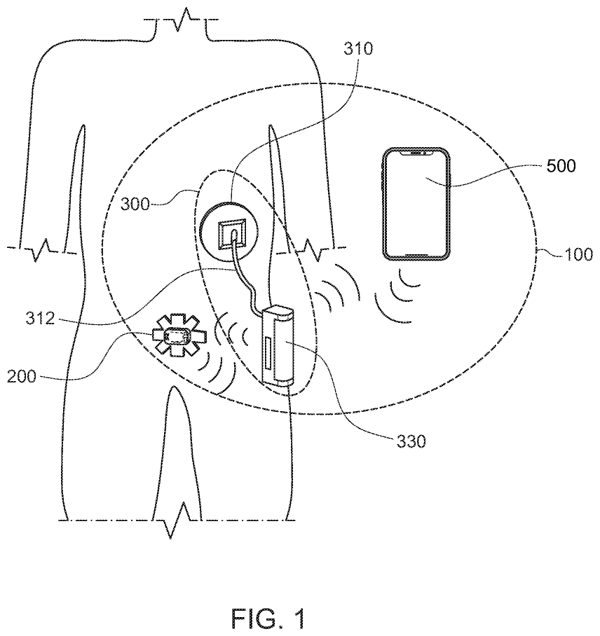

FIG. 1 is an illustration of a closed-loop home diuretic system 100 in accordance with an exemplary embodiment. As shown in FIG. 1, the closed-loop home diuretic system 100 comprises a urine output volume monitoring device 200, a diuretic administration device 300 which includes a subcutaneous injection site 310, a drug delivery tubing 312, a drug delivery and storage unit 330, and in some embodiments, a mobile device 500 with an application 510 (hereinafter simply referred to as “app 510”).

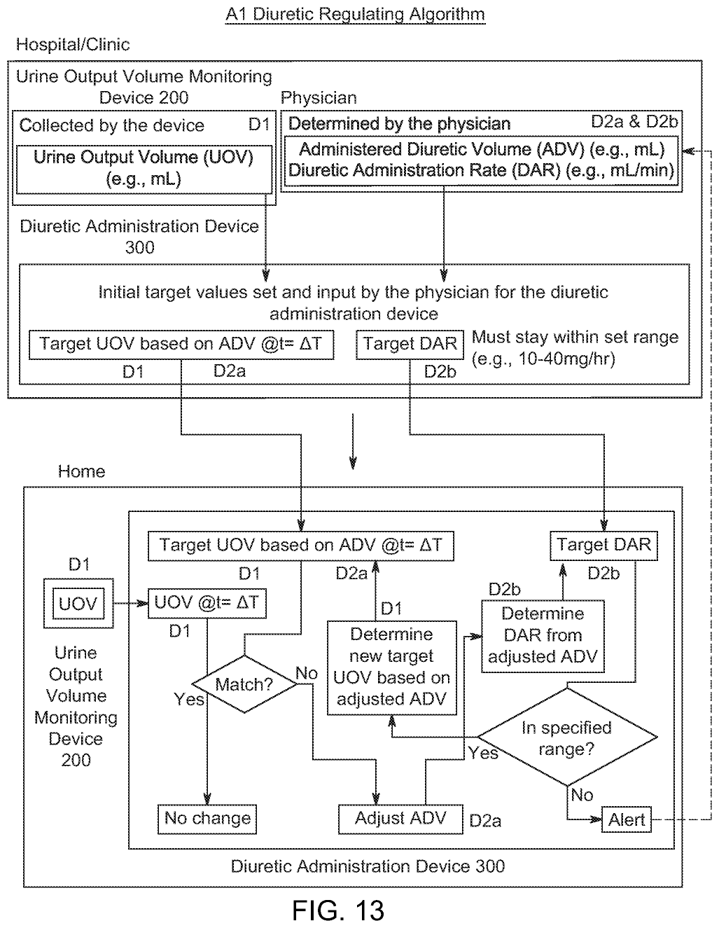

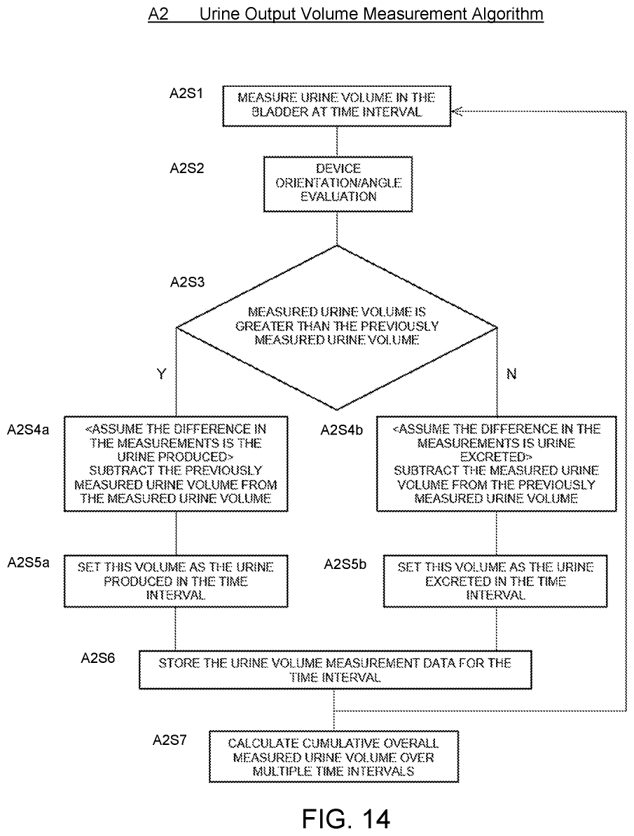

In accordance with an exemplary embodiment, the urine output volume monitoring device 200 detects and monitors the amount of urine in the bladder and calculates and stores urine output volume D1 using a urine output volume measurement algorithm A2 (see FIG. 14). Wireless communication hardware and technology such as Bluetooth built in the urine output volume monitoring device 200 send the urine output volume D1 to the diuretic administration device 300 and the mobile device 500. The mobile device 500 with the app 510 uses the urine output volume D1, existing target diuretic dose D2 (D2a, D2b) which, for example, have been recorded at a hospital or a clinic, and a diuretic regulating algorithm A1 (see FIG. 13) to determine a new target diuretic dose D2 and wirelessly sends the new target diuretic dose D2 to the diuretic administration device 300. Subsequently, the drug delivery and storage unit 330 delivers the diuretic according to the new target diuretic dose D2, to the subcutaneous injection site 310 by means of the drug delivery tubing 312.

In accordance with an exemplary embodiment, alternatively, the diuretic administration device 300 may use the urine output volume D1 directly from the urine output volume monitoring device 200 (i.e., bypassing the mobile device 500 with the app 510). In this alternative embodiment, the existing target diuretic dose D2 is stored within the diuretic administration device 300, and the diuretic regulating algorithm A1 executed by a build-in processor 321 of the diuretic administration device 300 determines the new target dose D2, and the drug delivery and storage unit 330 delivers the diuretic to the subcutaneous injection site 310 by means of the drug delivery tubing 312.

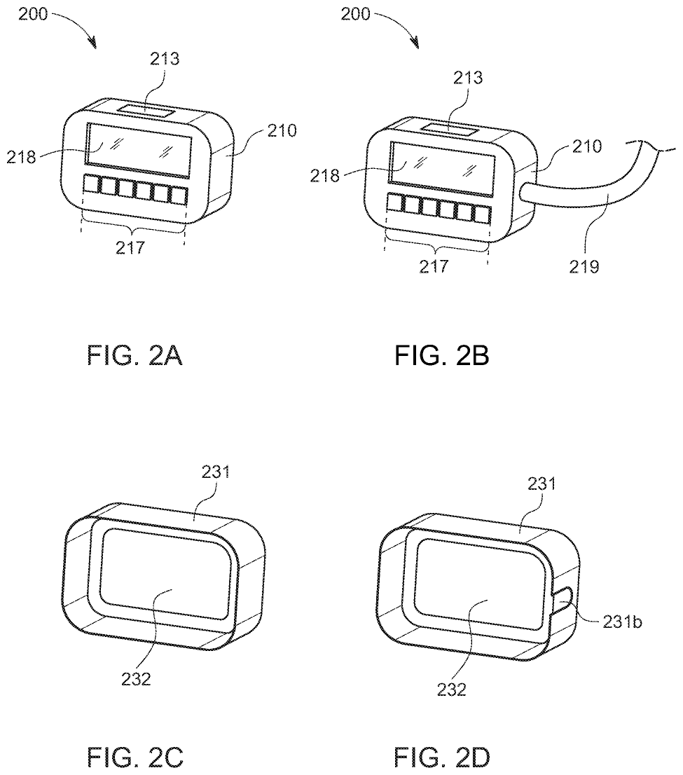

FIGS. 2A and 2B are perspective views of a monitor housing 210 for housing various components of the urine output volume monitoring device 200 in accordance with an exemplary embodiment. As shown in FIGS. 2A and 2B, a speaker 213, a control panel 217, and a display screen 218 are disposed along the outer surface of the monitor housing 210. FIG. 2B shows a variation of the exemplary embodiment with an electric cable 219 that may be used to physically connect to any auxiliary component such as a battery pack or other devices in the closed-loop home diuretic therapy system 100 if the wireless technology is not available.

FIGS. 2C and 2D are perspective views of a monitor housing holder 231 that the monitor housing 210 can be inserted and attached. In an exemplary embodiment, the inner perimeter dimensions of the monitor housing holder 231 is closely matched with the outer perimeter dimensions of the monitor housing 210 such that the monitor housing 210 can be snapped in firmly onto the monitor housing holder 231. The monitor housing holder 231, for example, may be made of plastic, elastic, or semi-elastic materials such as polycarbonate, acrylonitrile butadiene styrene (ABS), Pebax©, polyetheretherketone (PEEK), silicone, polyethylene, or polyurethane. FIG. 2D shows a variation of the exemplary embodiment with a slot 231b to allow for the electric cable 219 depicted in FIG. 2B to be passed through. The opening 232, which is an open window, has the perimeter dimensions that are close to the perimeter dimensions of the monitor housing holder 231 for better signal transmission from an ultrasound sensor 211 or an acoustic sensor 212 inside the monitor housing 210, the details of which will be described later.

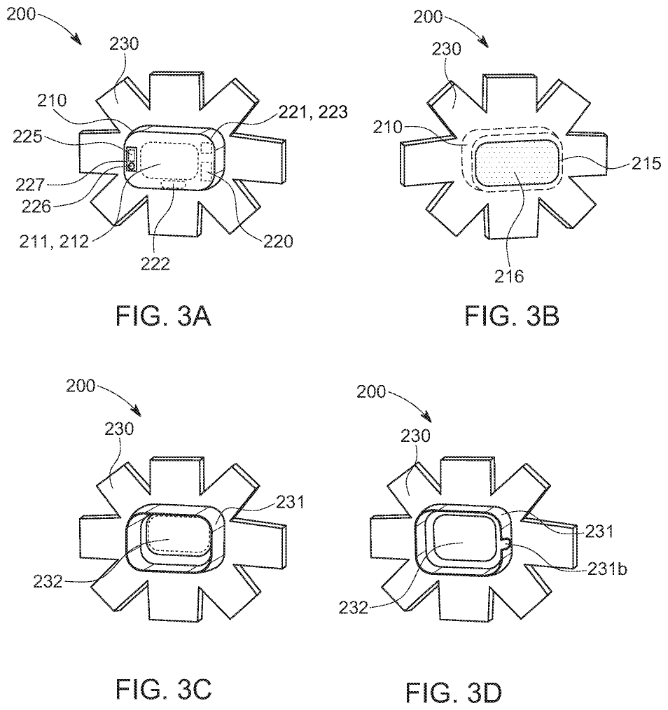

FIGS. 3A through 3D are perspective views of the urine output volume monitoring device 200 with an adhesive attachment 230 in accordance with an exemplary embodiment. FIGS. 3A and 3B show the adhesive attachment 230 directly on the monitor housing 210 whereas FIGS. 3C and 3D show the attachment 230 on the monitor housing holder 231. FIG. 3A is a view of the monitor housing 210 side opposite the skin-contact side, and FIG. 3B is the skin-contact side of the urine output volume monitoring device 200. The adhesive attachment 230 is thin, for example, 0.1 mm to 2 mm in thickness, and connected to the monitor housing 210 (FIGS. 3A and 3B) or to the monitor housing holder 231 (FIGS. 3C and 3D) such that the adhesive attachment 230 extends out from the outer profile of the monitor housing 210 or the monitor housing holder 231. The adhesive attachment 230 has an opening between the ultrasound sensor 211 and the skin-contact side of the monitor housing 210 or the monitor housing holder 231 so as to allow for better ultrasound signal transmission.

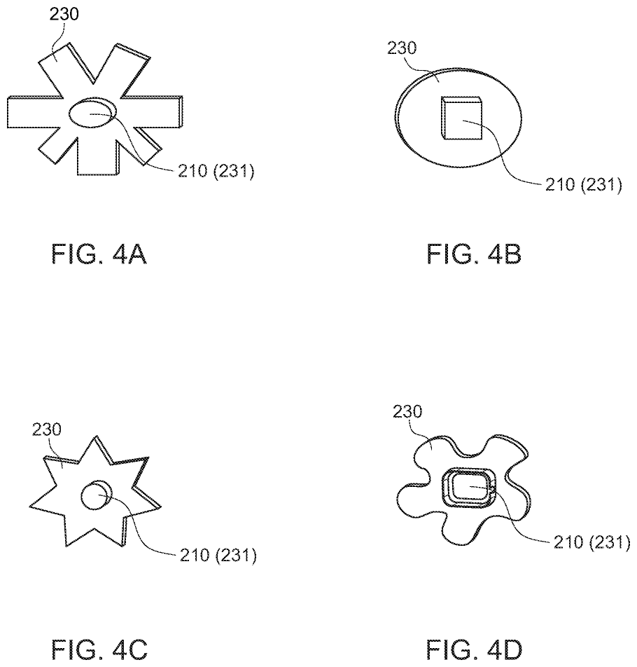

The adhesive attachment 230 is depicted with eight separate tabs in FIGS. 3A through 3D but it may, for example, have a contiguous outer periphery without any slits or openings, or have any number of openings to conform easier to the uneven surface of the skin when attached as illustrated in the schematics of FIGS. 4A through 4D. If the adhesive attachment 230 has a contiguous outer periphery without any slits or openings, the outer profile of the adhesive attachment 230 may be either smooth or uneven as shown in FIGS. 4B and 4D. If the adhesive attachment 230 is separated by slits or openings, each interrupted section is not required to have the same shape, length, or width as the other sections, as shown in FIGS. 4A and 4C. The monitor housing 210 that is depicted as a rectangular box with rounded corners in FIG. 3A, may have different outer profile shapes such as, for example, oval, square, or circular as shown in FIGS. 4A-4C, with dimensions, for example, a thickness of 1 mm to 30 mm and an overall circumscribing diameter of 2 cm to 30 cm.

In accordance with an exemplary embodiment, the urine output volume monitoring device 200, worn to be positioned anterior to the bladder on the lower abdomen, utilizes the ultrasound sensor 211 contained in the monitor housing 210 to send the ultrasound signals to detect the bladder wall. The monitor housing 210 also contains an accelerometer (e.g., a three-axis accelerometer) and/or a gyroscope 220 (hereinafter simply referred to as “accelerometer 220”) that can detect the orientation change from the change in the body position which may influence the measurement of urine volume in the bladder. A processor 221 that is built in the monitor housing 210 utilizes the data from the ultrasound sensor 211, the acoustic sensor 212, and/or the accelerometer 220 to estimate the urine in the bladder. The processor 221 then uses the series of data of urine in the bladder to determine the urine output volume over a specified time period. The urine output volume data stored in a memory 222 may be sent to other devices in the closed-loop home diuretic therapy system 100 by a network interface (I/F) 223 such as Bluetooth also built in the monitor housing 210.

In accordance with an exemplary embodiment, as shown in FIG. 3B, the monitor housing 210 may include a medium layer 215 to reduce or eliminate an air gap between the ultrasound sensor 211 and the contact area of the patient to enhance passing of ultrasound signals through human tissue. The medium layer 215 may contain fluid, absorb fluid, or allow fluid to pass through one or more fluid holes 216 to enhance its ability to reduce or eliminate an air gap between the ultrasound sensor 211 and the contact area of the patient. The medium layer 215 may be a replaceable component.

In accordance with an exemplary embodiment, as shown in FIG. 3A, the urine output volume monitoring device 200 or the monitor housing 210 has a power source 225 which may be, for example, a rechargeable battery that can be recharged by connecting to an external power source by means of a power cord or a replaceable battery. The monitor housing 210 may have a sensor charging port 226 for the power cord, a monitor housing access panel 227 that can be opened and closed when replacing the battery, or both (226 and 227). The locations and configurations of the sensor charging port 226 and the monitor housing access panel 227 are not limited to those depicted in FIG. 3A.

In accordance with an exemplary embodiment, the urine output volume monitoring device 200 utilizes the ultrasound sensor 211 that can detect the bladder wall when it is placed on the skin of the lower abdomen near the bladder. By detecting the balder wall and determining the amount of urine in the bladder, a urine output volume measurement algorithm (e.g., A2 shown in FIG. 14) that is executed by the processor 221 of the urine output volume monitoring device 200, can determine the total urine output volume over a certain time period. The urine output volume is stored in the memory 222 of the urine output volume monitoring device 200 and can be sent to the other devices in the closed-loop home diuretic therapy system 100 by wireless technology such as Bluetooth using the network interface 223 of the urine output volume monitoring device 200.

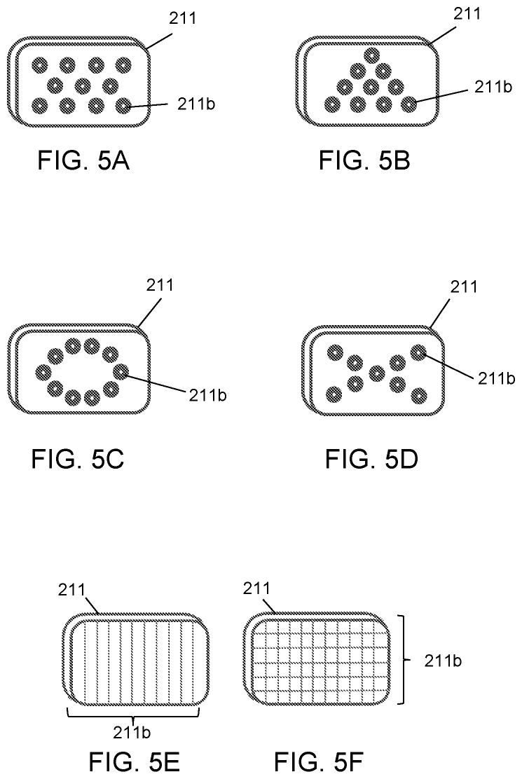

FIGS. 5A-5F illustrate examples of the ultrasound sensor 211, which may be an array of sensor elements 211b to detect and capture the entire profile of the bladder in all anatomical directions. The sensor elements 221b may be arranged in one to ten rows by one to ten columns (e.g., 1×1, 2×3, 3×2, 3×3, 5×10, 8×6, etc.). The array is not limited to possessing the same number of sensors in each row or column. The array may include any number of sensor elements 211b in each row and column.

In one embodiment, as shown in FIG. 5A, the multiple sensor elements 211b may be arranged in three rows configured 4-3-4 in which the number of rows and number of sensor elements 211b in each row, for example, may range from one to one thousand wherein the number of sensor elements 211b in each row may or may not be the same. In another embodiment, as shown in FIG. 5B, the sensor elements 211b may be arranged in a triangle in which the number of sensor elements 211b forming the triangle, for example, may range from three to forty wherein the number of sensor elements 211b in each subsequent row may or may not be incremented by one. In another embodiment, as shown in FIG. 5C, the sensor elements 211b are arranged in a circular or oval pattern in which the number of sensor elements 211b forming the circular or oval pattern, for example, may range from four to forty wherein the sensor elements 211b may be positioned on the inside of the circular or oval pattern. In yet another embodiment, as shown in FIG. 5D, the sensor elements 211b may form an X shape in which the number of the sensor elements 211b, for example, may range from three to forty wherein the angle that forms the X shape and the angular orientation of the X shape may be between 0° and 180°.

In one embodiment, as shown in FIGS. 5E and 5F, the sensor elements 211b may be diced from a block of material. The number of rows and columns of the diced elements, for example, may range from one to one thousand.

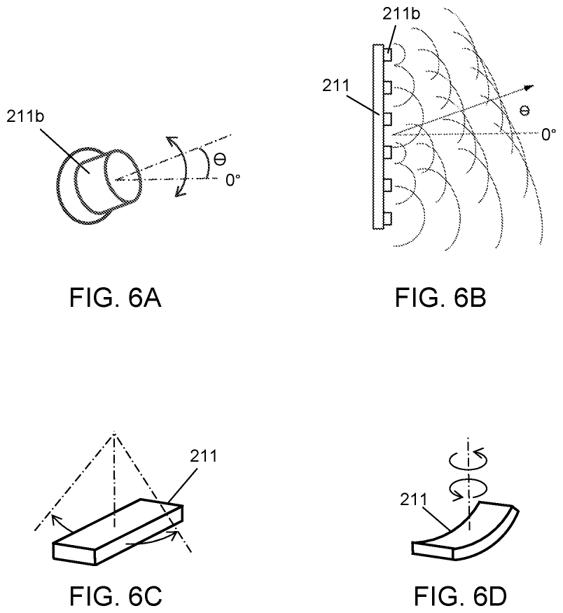

In accordance with an exemplary embodiment, the ultrasound sensor 211 may change the ultrasound signal directions to expand the scanning region to detect the bladder wall. The ultrasound signal directions may be altered by changing the angle of the sensor elements 211b as shown in FIG. 6A. The ability to change the angle of sensor elements 211b may reduce the number of total sensor elements 211b required to detect the bladder wall in different anatomical directions. Different angles of sensor elements 211b may be preset and be passive for the individual sensor elements 211b or may be actively controlled by the processor 221.

Alternatively, as shown in FIG. 6B, the ultrasound sensor 211 may utilize a phased array scanning approach in which the processor 221 electronically steers the ultrasound signals from the multiple sensor elements 211b to point in different directions without physically moving the sensor elements 211b.

In another embodiment, the ultrasound sensor 211, which is an array of sensor elements 211b, may automatically rotate in rocking motion as shown in FIG. 6C or in rotational motion as shown in FIG. 6D to allow for 3D scanning. The ultrasound sensor 211 may be convexly curved towards the skin-contact side as depicted in FIG. 6D to enhance the field of view.

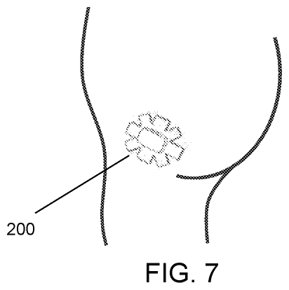

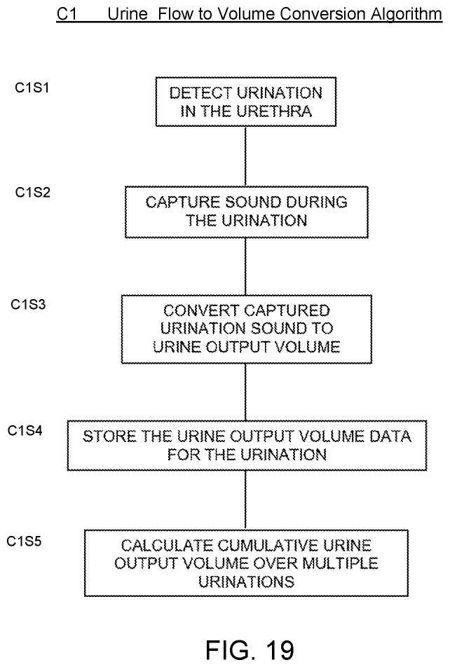

Alternatively, the urine output volume monitoring device 200 may utilize the acoustic sensor 212 that can detect the magnitude of urine flow inside the urethra when it is placed on the skin near the perineum as shown in FIG. 7. By detecting the amount of urine flow inside the urethra and recording it, a urine flow to volume conversion algorithm C1 shown in FIG. 19 and executed by the processor 221 of the urine output volume monitoring device 200, can determine the total urine output volume over a certain time period. The urine output volume D1 is stored in the memory 222 of the urine output volume monitoring device 200 and can be sent to other devices in the closed-loop home diuretic therapy system 100 by wireless technology such as Bluetooth using the network interface 223 of the urine output volume monitoring device 200.

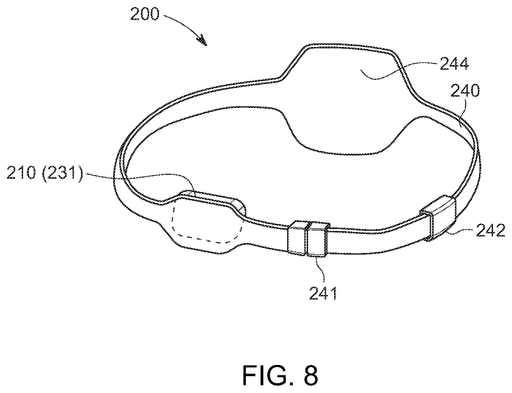

FIG. 8 is a perspective view of the urine output volume monitoring device 200 configured with an adjustable belt strap 240 in accordance with an exemplary embodiment. The monitor housing 210 is attached on the inner side of the adjustable belt strap 240 by means of the monitor housing holder 231 to contact the skin of the lower abdomen, where the ultrasound sensor 211 inside the monitor housing 210 is utilized for the urine output volume monitoring.

The adjustable belt strap 240 has a belt connector 241 that can be connected and disconnected by the wearer or an assisting person to put on or take off the adjustable belt strap 240. The adjustable belt strap 240 also has a belt adjuster 242 that can be utilized to adjust the length of the adjustable belt strap 240 to fit varying body shapes and sizes. A back support 244 provides stability and comfort for the wearer of the adjustable belt strap 240.

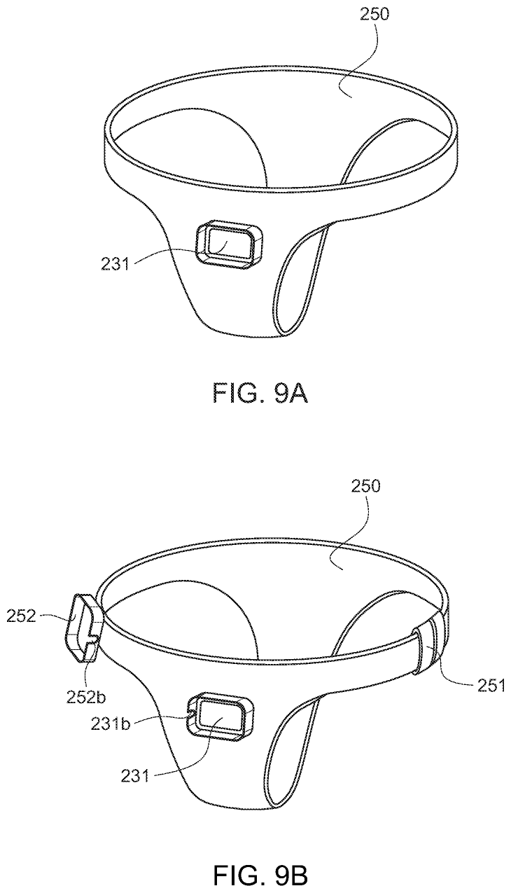

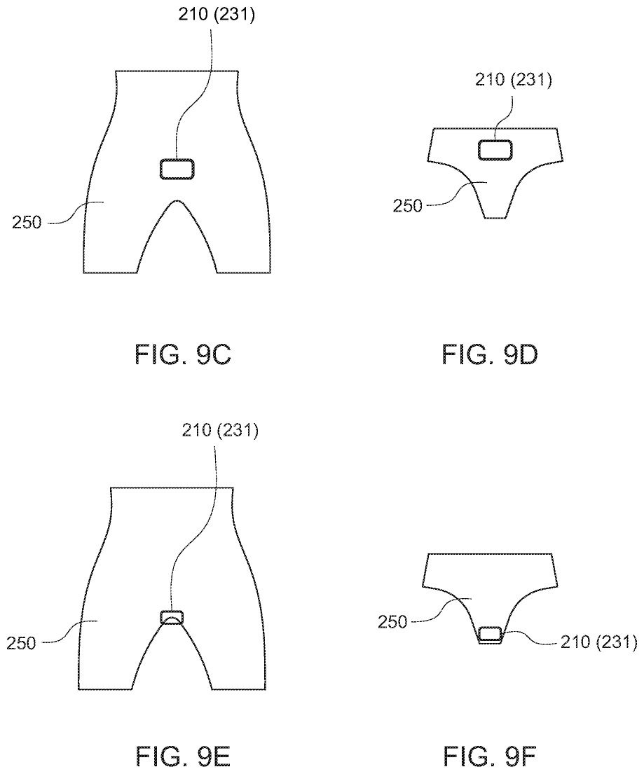

Alternatively, the urine output volume monitoring device 200 may be a part of an undergarment 250 as depicted in FIGS. 9A through 9F. The monitor housing holder 231 may be attached to the undergarment 250 such that the monitor housing 210 can be attached. FIG. 9A is a perspective view of the undergarment 250 that may be elastic and may come in different sizes for varying body types of the patient. The undergarment 250 may have a connector and adjuster 251 as depicted in FIG. 9B such that the waist dimension can be adjusted to fit varying body shapes and sizes. The undergarment 250 may also have one or more additional holders 252 for any auxiliary component(s) such as a battery pack or other devices in the closed-loop home diuretic therapy system 100. The slot 252b on the additional holder 252 functions the same as the slot 231b of the monitor housing holder 231 depicted in FIG. 2D to allow for a cable such as the electric cable 219 depicted in FIG. 2B to be passed through. The material of the undergarment 250, for example, may be polyethylene, polypropylene, polyester, neoprene, or cotton. FIGS. 9C and 9D show schematic illustrations of the undergarment 250 in different configurations, one showing longer support along the thighs and the waist and the other showing the similar shape as the undergarment 250 in FIGS. 9A and 9B.

In one embodiment, the urine output volume monitoring device 200 can be placed near the perineum as shown in FIGS. 9E and 9F so that the acoustic sensor 212 can detect the magnitude of urine flow inside the urethra.

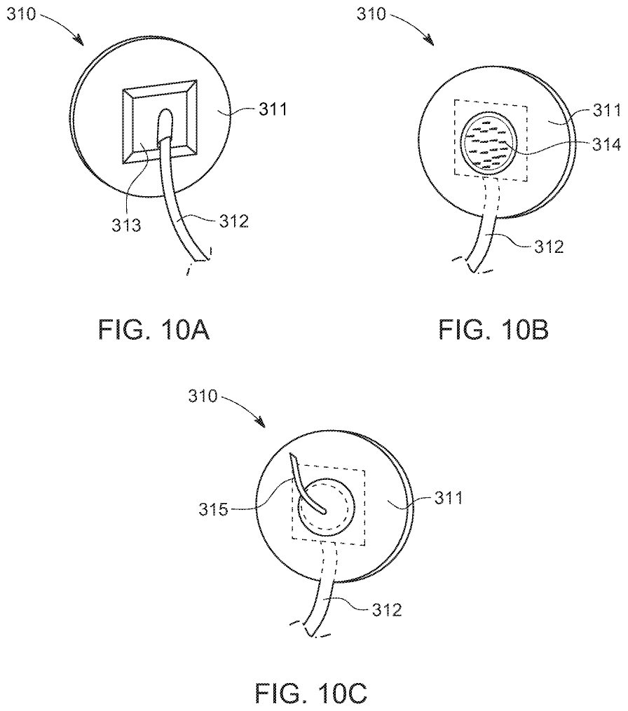

In accordance with an exemplary embodiment, FIGS. 10A and 10B are perspective views of the subcutaneous injection site 310 of the diuretic administration device 300 illustrating a delivery site attachment 311, the drug delivery tubing 312, a tubing connector 313, and an array of microneedles 314.

FIG. 10A shows the side of the subcutaneous injection site 310 opposite the skin-contact side, whereas FIG. 10B shows the skin-contact side of the subcutaneous injection site 310. As shown in FIG. 10A, the drug delivery tubing 312 is connected to the tubing connector 313. The delivery site attachment 311 is thin, for example, 0.1 mm to 2 mm in thickness, and extends out of the outer profile of the tubing connector 313. The delivery site attachment 311 and the tubing connector 313 are arranged such that the surface of the array of microneedles 314 directly contacts the skin and the delivery site attachment 311 is not between the surface of the array of microneedles 314 and the skin as shown in FIG. 10B. The array of microneedles 314 subcutaneously delivers the diuretic pumped by the drug delivery and storage unit 330 by means of the drug delivery tubing 312 and the tubing connector 313. Alternatively, as shown in FIG. 10C, the array of microneedles 314 may be replaced by a cannula 315 to be placed under the skin for subcutaneous injection. The delivery site attachment 311 is depicted as a circular sheet with a contiguous outer periphery in FIGS. 10A through 10C but may, for example, possess one to twelve slits or openings to conform easier to the uneven surface of the skin when attached. If the delivery site attachment 311 has a contiguous outer periphery without any slits or openings, the outer profile of the delivery site attachment 311 may be either smooth or uneven. If the delivery site attachment 311 is separated by slits or openings, each interrupted section is not required to have the same shape, length, or width as the other sections. The tubing connector 313 that is depicted as a square box in FIG. 10A, may have different outer profile shapes such as, for example, rectangular, circular, or oval and the following dimensions, for example: a thickness of 1 mm to 15 mm and an overall circumscribing diameter of 1 cm to 5 cm. The same configuration as the adhesive attachment 230 illustrated in FIGS. 3A through 3D and FIGS. 4A through 4D can apply to the delivery site attachment 311.

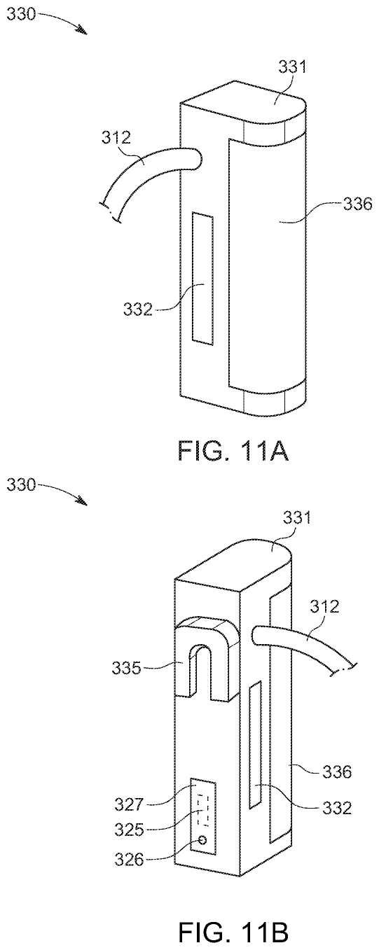

In accordance with an exemplary embodiment, FIGS. 11A and 11B are perspective views of the drug delivery and storage unit 330 of the diuretic administration device 300 with a pumping unit 331 and a drug cartridge 336 engaged and connected.

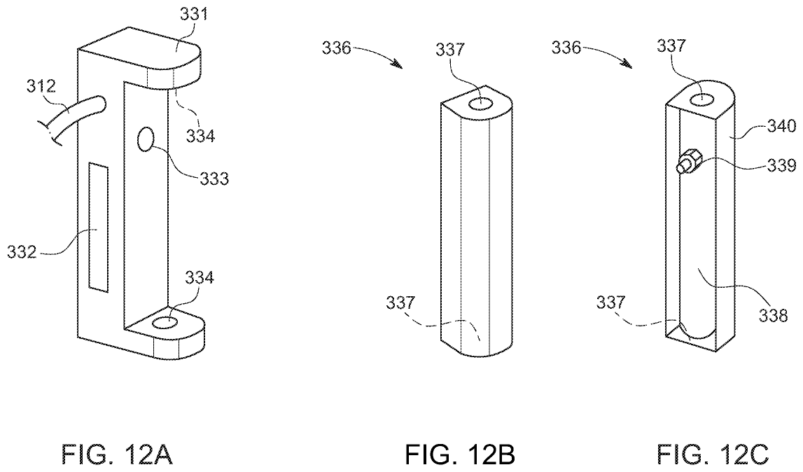

Also in accordance with an exemplary embodiment, FIGS. 12A through 12C are perspective views of the drug delivery and storage unit 330 of the diuretic administration device 300 with the pumping unit 331 and the drug cartridge 336 disengaged and separated.

FIG. 11A shows the front (i.e., the drug cartridge 336 side) view whereas FIG. 11B shows the back (i.e., the pump attachment 335 side) view of the drug delivery and storage unit 330. As shown in FIGS. 11A and 11B, the drug delivery tubing 312 connects to the pumping unit 331. The pumping unit 331 also has a manual control panel and indicator 332 and a pump attachment 335 which may be a clip, for example, that can be utilized to attach the drug delivery and storage unit 330 to the user. In accordance with an exemplary embodiment, the pumping unit 331 also has a pumping unit power source 325 which may be, for example, a rechargeable battery that can be recharged by connecting to an external power source by means of a power cord or a replaceable battery. The pumping unit 331 has a pumping unit charging port 326 for the power cord, a pumping unit access panel 327 that is opened when replacing the battery, or both (326 and 327). It is noted that the locations and configurations of the pumping unit charging port 326 and the pumping unit access panel 327 are not limited to those depicted in FIG. 11B.

Also in accordance with an exemplary embodiment, FIGS. 12A through 12C are perspective views of the drug delivery and storage unit 330 of the diuretic administration device 300 with the pumping unit 331 and the drug cartridge 336 disengaged and separated. FIGS. 12B and 12C show the drug cartridge 336 disengaged and separated from the pumping unit 331. FIG. 12C shows a cartridge shell 340 encasing a drug compartment 338. A male drug delivery connector 339 shown in FIG. 12C protrudes out of the drug compartment 338 and connectable to a female drug delivery connector 333 on the pumping unit 331 shown in FIG. 12A, such that the pumping mechanism that is built in the pumping unit 331 can draw the drug out of the drug compartment 338 of the drug cartridge 336 when connected.

Cartridge connectors 337 on the drug cartridge 336 depicted in FIGS. 12B and 12C correspond to cartridge receivers 334 of the pumping unit 331 depicted in FIG. 12A. The cartridge connectors 337 may be, for example, a spring-loaded tabs or latches whereas the corresponding cartridge receivers 334 may be indentations to allow the tabs or latches to snap in to secure the drug cartridge 336 in the pumping unit 331.

FIG. 13 is a flowchart of the diuretic regulating algorithm A1 for the closed-loop home diuretic therapy system 100 in accordance with an exemplary embodiment. The algorithm uses the urine output volume (UOV) D1, the administered diuretic volume (ADV) D2a, and the diuretic administration rate (DAR) D2b to regulate the diuretic to be administered. For any specified time period (denoted as t=ΔT in FIG. 13), the UOV D1 should correspond to the ADV D2a given to the patient.

The closed-loop home diuretic therapy system 100, for example, may be provided at the hospital prior to discharge of HF patients as a transition from the IV diuretic therapy and be prescribed by a physician for continued home use. This period provides an opportunity to gather the patient-specific information on the UOV D1, ADV D2a, and DAR D2b using which a physician can determine the target ADV D2a and DAR D2b for the patient to produce the appropriate amount of UOV D1 for a certain time period (e.g., six hours which is frequently used by physicians for HF patients). The DAR D2b is used because the ADV D2a by itself does not provide the information necessary to determine the diuretic efficacy monitored by the UOV D1. How much diuretic is administered over a given time period (D2b) and how much urine was produced and excreted (D1) for a given amount of diuretic administered (D2a) provide the essential information for regulating the diuretic for HF patients. DAR D2b should also be in a certain range set by the physician for patient safety reasons.

As shown in FIG. 13, the UOV D1 produced as the result of the ADV D2a given to the patient is consistently compared against the target values over a specific time period such that ADV D2a can be adjusted and a new DAR D2b determined as necessary to maintain the balance of the UOV D1 corresponding to the ADV D2a and DAR D2b. Prior to using the new DAR D2b determined by the adjusted ADV D2a, the new DAR D2b is checked against the target range of the DAR D2b set by the physician such that the patient would be alerted by the device if the DAR D2b would go outside of the range. In such a scenario, the patient may contact the physician or the device may send the alert directly to the physician.

FIG. 14 is a flowchart of the urine output volume measurement algorithm A2 for the closed-loop home diuretic therapy system 100 in accordance with an exemplary embodiment. The algorithm is executed in the urine output volume monitoring device 200 and uses multiple urine measurements inside the bladder for a given time interval to obtain the cumulative urine output volume D1 over a certain time period.

In accordance with an exemplary embodiment, the first step A2S1 of the urine output volume measurement algorithm A2 is performed by the urine output volume monitoring device 200 to measure the urine in the bladder at a set time interval, for example, anywhere from one minute to one hour. The second step A2S2 utilizes the measurement adjustment for device orientation algorithm B1 or the user alarm for device orientation algorithm B2 to consider the orientation change of the bladder due to different body positions. Details of these algorithms are described further below.

Also in accordance with an exemplary embodiment, the third step A2S3 of the urine output volume measurement algorithm A2 is a decision point to determine whether the measured urine volume in the bladder for the time interval is greater than the previously measured urine volume in the bladder. If the measured urine volume is greater than the previously measured urine volume in the bladder, then an assumption is made that the difference in the urine volume is the urine that was produced and accumulated in the bladder in the time interval (A2S4a), and the urine volume is set as the urine produced in the time interval (A2S5a). Otherwise (e.g., when the measured urine volume is less than or equal to the previously measured urine volume), an assumption is made that the difference in the urine volume is the urine that was excreted from the bladder in the time interval (A2S4b), noting that the amount zero would be recorded if the measured urine volume is equal to the previously measured urine volume, and the urine volume is set as the urine excreted in the time interval (A2S5b). The urine volume data is then stored (A2S6), and the cumulative urine output volume can be calculated by adding the multiple time intervals for a certain time period (A2S7), for example, anywhere from five minutes to six hours.

In one embodiment, the urine output volume measurement algorithm A2 is executed based on an output from the accelerometer 220. For example, the urine output volume monitoring device 200 executes the algorithm A2 when acceleration is not detected by the accelerometer 220. Additionally, the urine output volume monitoring device 200 may refrain from executing the algorithm A2 during a period in which high acceleration is detected. In other words, the urine output volume monitoring device 200 controls the ultrasound sensor 211 to not emit ultrasound signals during that period and/or when acceleration is detected. With this configuration, power consumption by the ultrasound sensor 211 can be reduced, and the volume of the bladder can be reliably determined.

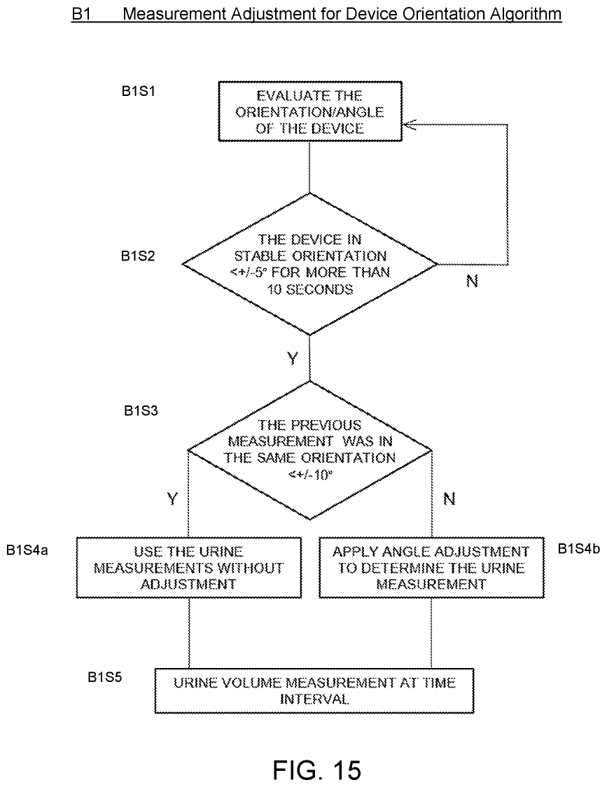

FIG. 15 is a flowchart of the measurement adjustment for device orientation algorithm B1 for the closed-loop home diuretic therapy system 100 in accordance with an exemplary embodiment. This algorithm is utilized in the second step A2S2 of the urine output volume measurement algorithm A2 described above. In accordance with an exemplary embodiment, the first step B1S1 of the measurement adjustment for device orientation algorithm B1 is performed by the urine output volume monitoring device 200 to evaluate the device's orientation or angle using the accelerometer 220 built in the monitor housing 210 of the urine output volume monitoring device 200. The accelerometer 220 can sense the orientation or angle of the device with respect to the horizontal surface which may be defined as its zero or base position, thus being able to detect any relative changes in the orientation or angle from such position.

The second step B1S2 of the measurement adjustment for device orientation algorithm B1 is a decision point to determine whether the urine output volume monitoring device 200 has been in stable orientation for measurement without deviating from the zero or base position by, for example, less than plus or minus 2° to 10° and, for example, for more than five to fifteen seconds. If the device is deemed to be not stable for measurement, then the step B1S2 is repeated until it is deemed stable based on the deviation criteria of B1S2. If the device is deemed stable for measurement, then the algorithm proceeds to the next step B1S3 which is another decision point to determine whether the previous measurement was taken in the same relative orientation or angle by, for example, less than plus or minus 5° to 20°. If the previous measurement was deemed to be taken in the same relative orientation or angle, then no adjustment is made and the algorithm sets the measurement as is (B1S4a). If the previous measurement was deemed to be taken in different relative orientation or angle based on the deviation criteria of B1S3, then an adjustment is made on the measurement based on the amount of angle deviation (B1S4b). The adjustment is based on the body position's effect on how the ultrasound sensor 211 detects the bladder and the urine which may be, for example, derived from generally known anatomical bladder positions for different body positions, derived from the data from the ultrasound sensor 211, or combination of both. Once the measurement is set (B1S5), it is used in the third step A2S3 of the urine output volume measurement algorithm A2.

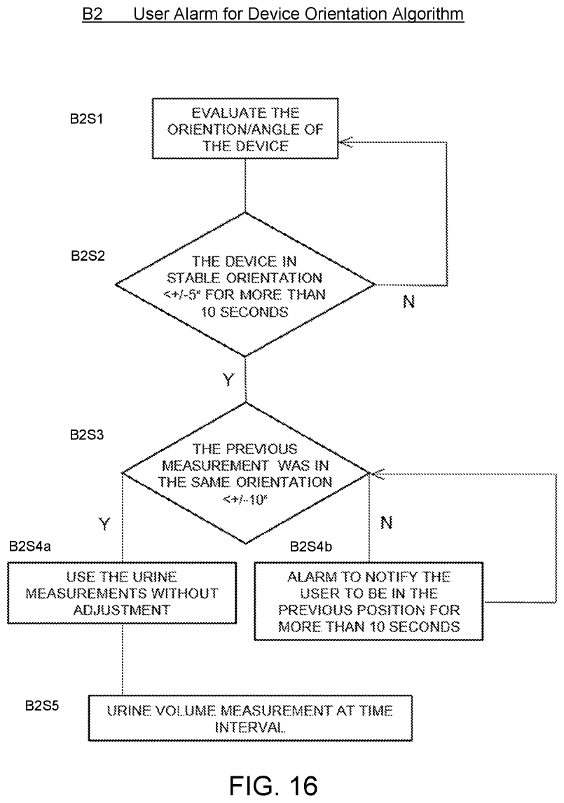

FIG. 16 is a flowchart of the user alarm for device orientation algorithm B2 for the closed-loop home diuretic therapy system 100 in accordance with an exemplary embodiment. This algorithm is an alternative to the measurement adjustment for device orientation algorithm B1 utilized in the second step A2S2 of the urine output volume measurement algorithm A2. The steps B2S1, B2S2, B2S3, and B2S4a of the user alarm for device orientation algorithm B2 are the same as steps B1S1, B1S2, B1S3, and B1S4a of the measurement adjustment for device orientation algorithm B1, respectively. The difference is in the step after the decision point B2S3, in the situation when the previous measurement was deemed to be taken in different relative orientation or angle. In such a case, the user alarm for device orientation algorithm B2 triggers an alarm for the urine output volume monitoring device 200 to alert the user to be in the same stable body position as when the previous measurement was taken (B2S4b). The instructions may be displayed on or dictated by the mobile device 500 or the display screen 218 or the speaker 213 of the urine output volume monitoring device 200. The cycle of steps B2S3 and B2S4b is repeated until the measurement is deemed acceptable by the criteria of B2S3 and no more measurements are required (B2S4a). Once the measurement is set (B2S5), it is used in the third step A2S3 of the urine output volume measurement algorithm A2.

In one embodiment, the measurement of the urine volume and the evaluation of the device orientation/angle in the steps A2S1 and A2S2 of FIG. 14 may be performed using machine learning and/or sensor fusion techniques. For example, the closed-loop home diuretic therapy system 100 employs a neural network (i.e., supervised machine learning) which uses acceleration readings and/or gyroscope readings in addition to ultrasound measurements (input layer) to estimate the bladder volume (output layer) at each time point. Incorporating the accelerometer and gyroscope measurements can improve the accuracy of the bladder volume estimates generated by the neural network. The neural network can account for patient orientation and motion to generate more accurate volume estimates. In turn, improved bladder volume estimates would better inform the system when deciding on the amount of diuretic to deliver. To use this approach, the neural network is trained with the sensor data (i.e., ultrasound, accelerometer, and/or gyroscope) before deploying the neural network model on the closed-loop home diuretic therapy system 100.

Additionally, the closed-loop home diuretic therapy system 100 can employ an unsupervised machine learning algorithm, e.g., k-means clustering, to automatically identify locations of the bladder in the volumetric ultrasound measurements and, in turn, estimate the bladder volume. This bladder estimation occurs at every acquired time point, e.g., once a minute. In this implementation, the accelerometer and/or gyroscope measurements can be used to indicate error level of these ultrasound measurements as greater motion is associated with higher error. A Kalman filter is one sensor fusion technique to minimize the volume error due to patient motion. Kalman filters “denoise” measurements by informing current time points with previous time point measurements according to the amount of error in the current time point versus the previous time point.

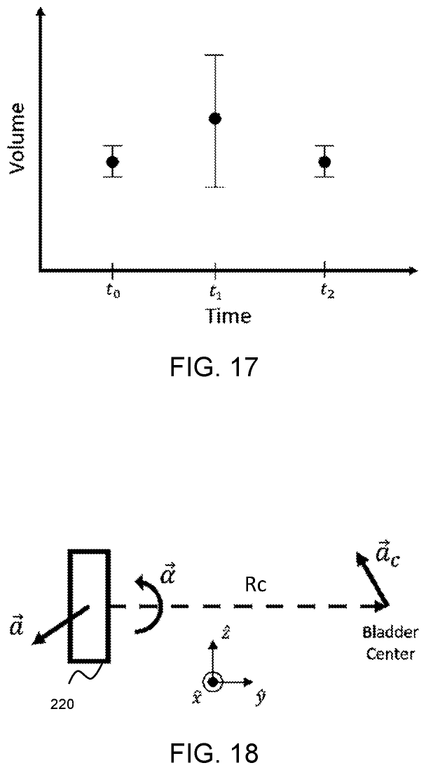

An algorithm for determining the error level of measured bladder volume using the accelerometer and gyroscope measurements is described with reference to FIGS. 17 and 18. In one embodiment, the urine output volume monitoring device 200 employs a Kalman filter to estimate the current state of the bladder defined by its volume, V, and rate of change of the volume in time, \dot{V}. Kalman filtering reduces the effects of noise and patient motion by reconciling estimations and actual measurements. The “filtered” bladder volume at time t can be expressed as the combination of the modeled volume, V_{Mod}, and the measured volume, V_{Meas}:

$$V ( t ) = ( 1 - K ( t ) ) {V}_{Mod} ( t ) + K ( t ) {V}_{M e a s} ( t ) \qquad [ Math . 1 ]$$

where K(t) is the Kalman gain governing how much the modeled versus measured volumes should play into calculating the “filtered” volume. The accelerometer and gyroscope readings can be used to help determine the value of K at all time points. V_{Mod} is evaluated by projecting the previous volume estimate using the estimated volume rate of change:

$${V}_{Mod} ( t ) = {V}_{Mod} ( t - 1 ) + \dot{V} ( t - 1 ) Δ t \qquad [ Math . 2 ]$$

In FIG. 17, volume measurements are estimated at three time points (t₀, t₁, t₂). There is no motion at t₀ and t₂, but there is motion during t₁. In FIG. 17, the volume estimates are represented with circles, and the error in the measurements is represented using the vertical error bars. A larger error bar is shown because of the motion at t₁. In one embodiment, the size of these error bars is determined using the readings from the accelerometer and gyroscope. The Kalman filter will then automatically infer that the dramatic change in volume measured at t₁ is likely due to error introduced by the patient moving. This will result in a low value of K at t₁, meaning the modeled volume will be more relied on.

Here, a method for determining the size of error bars using the accelerometer and gyroscope is explained with reference to FIG. 18. The method first estimates the acceleration of the sensor relative to the bladder using kinematic equations, then approximates the displacement of the bladder relative to the sensor using the relative acceleration. The method uses that displacement to estimate the volume uncertainty. The accelerometer and gyroscope provide time-resolved measurements of the sensor's 3D linear and angular acceleration, [symbol] (t) and [symbol] (t), respectively. The objective is to determine the acceleration at the center of the bladder, \vec{a}_{c}(t), in reference to the sensor. The bladder center is a distance of R_{c} away from the sensor.

The coordinate system used is shown in FIG. 18 with the y-axis being normal to the sensor face. The linear and angular accelerations can be written as their separate x, y, z components:

$$\vec{a} = < {a}_{x} , {a}_{y} , {a}_{z} > \qquad [ Math . 3 ]$$

$$\vec{α} = < {α}_{x} , {α}_{y} , {α}_{z} > \qquad [ Math . 4 ]$$

The ultrasound sensor 211 scans the bladder over a period of time, T_{acq}. During this time, the sensor will undergo linear and angular acceleration. The motion during T_{acq} that is estimated as the root mean square (RMS) evaluated in time across the duration of T_{acq} for each component can be expressed as follows:

$${\vec{a}}^{R M S} = RMS ( \vec{a} ( t ) ) \qquad [ Math . 5 ]$$

$${\vec{α}}^{R M S} = RMS ( \vec{α} ( t ) ) \qquad [ Math . 6 ]$$

Provided that [symbol] and [symbol] are representative of constant acceleration with initial linear and angular velocities equal to zero, \vec{a}_{c} can be expressed as the summation of linear and angular acceleration effects:

$${\vec{a}}_{c} = {\vec{a}}^{R M S} + [ {R}_{c} {α}_{x}^{R M S} \hat{z} - {R}_{c} {α}_{z}^{R M S} \hat{x} ] \qquad [ Math . 7 ]$$

Using the model of constant acceleration with zero initial velocity, the displacement of the bladder observed by the sensor, [symbol] , can be evaluated, after integrating over the duration T_{acq}:

$$\vec{d} = < {d}_{x} , {d}_{y} , {d}_{z} > = \frac{Δ {T}_{a c q}^{2}}{2} {\vec{a}}_{c} \qquad [ Math . 8 ]$$

The uncertainty due to sensor motion is expected to be proportional to the absolute product of all displacement components:

$${σ}_{m} ∝ \vert {d}_{x} {d}_{y} {d}_{z} \vert \qquad [ Math . 9 ]$$

where σₘ serves as the error bars around the estimated bladder volume at each time point. For the purposes of applying the Kalman filter, it is enough to know how Um compares across different time points, e.g., σₘ(t₀) versus σₘ (t₁). Knowing the proportional relationship of Um is sufficient for this purpose.

FIG. 19 shows the process of the urine flow to volume conversion algorithm C1 in which the first step C1S1 is to detect the onset of urine flow or urination by the acoustic sensor 212. After the onset of urine flow or urination has been detected, in step C1S2, the urine flow sound throughout the duration of urination is captured by the acoustic sensor 212. In the following step C1S3, the urine flow sound information is converted to volume based on the flow rate and the duration analyzed by the acoustic sensor 212. The urine volume data is then stored in step C1S4, and the cumulative urine output volume can be calculated by adding the multiple urinations for a certain time period in step C1S5, for example, anywhere from five minutes to six hours.

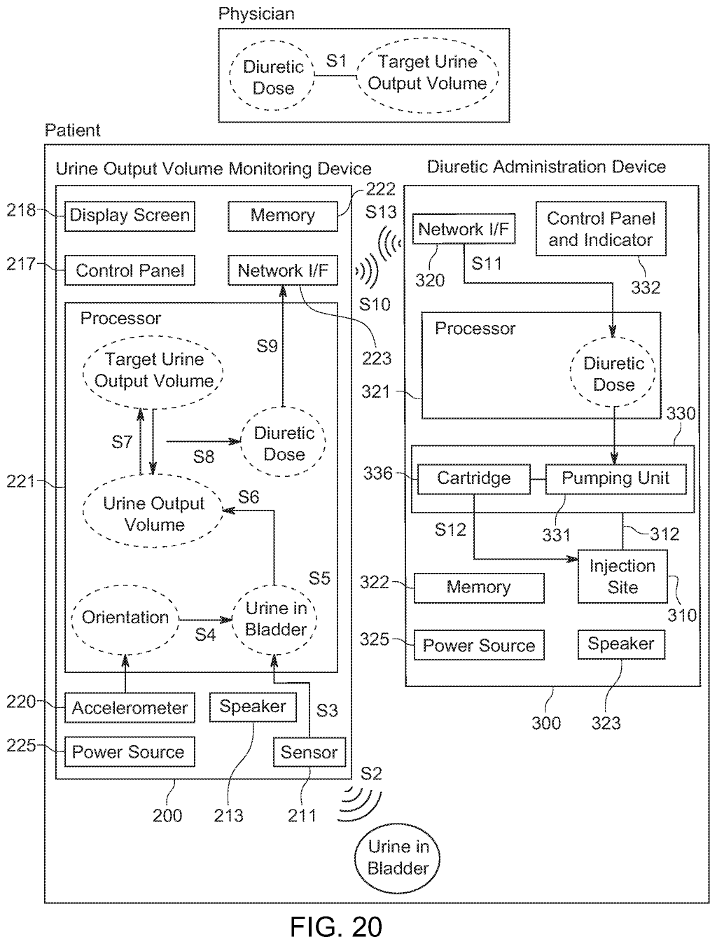

Now with reference to FIGS. 20 through 23, the operation of the closed-loop home diuretic therapy system 100 for determining and updating the diuretic dose to be administrated based on the target and measured urine output volume is described.

In an exemplary embodiment, the urine output volume monitoring device 200 may use its built-in processor 221 to determine the appropriate diuretic dose and send the diuretic dose data to the diuretic administration device 300, and diuretic is administered to the patient by means of the drug delivery and storage unit 330 delivering the diuretic to the subcutaneous injection site 310 through the drug delivery tubing 312. FIG. 20 shows the urine output volume monitoring device 200 and the diuretic administration device 300 that communicate with each other according to this embodiment. FIG. 20 depicts how the information determined by the physician may be initially used to set up the closed-loop home diuretic therapy system 100 and how the subsequent information and data may flow through the system 100.

In an exemplary embodiment, the urine output volume monitoring device 200 comprises the processor 221, the memory 222, the ultrasound sensor 211, the accelerometer 220, the speaker 213, the power source 225, the control panel 217, the display screen 218, and the network interface 223. The urine output volume monitoring device 200 may comprise any other component not shown in FIG. 20, e.g., the acoustic sensor 212 as described above. FIGS. 2A and 3A show an example of the arrangement of those hardware components.

The ultrasound sensor 211 emits ultrasound signals towards the bladder of the patient and detects the bladder wall thereof. For example, the accelerometer 220 is a three-axis accelerometer that detects the orientation of the urine output volume monitoring device 200. As described above, the accelerometer 220 may include a gyroscope. To measure device orientation, an inclinometer, a tilt sensor, and/or an inertial measurement unit (IMU) may be used in addition to or instead of the accelerometer 220. The speaker 213 functions to alert the user with sounds when necessary and optionally to generate responsive sounds when different actions are performed by the user through the control panel 217. The power source 225 is, e.g., a battery, which supplies power to the components of the urine output volume monitoring device 200. The control panel 217 may have buttons or switches for the user to input data and adjust settings of the urine output volume monitoring device 200. The display screen 218 may display the data, settings, and any functionally relevant information including warnings. The display screen 218 may also possess touch screen capabilities such that the selection can be made by touching the screen. The network interface 223 is a wireless network interface controller that communicates with other external devices such as the diuretic administration device 300 according to wireless communication protocols, e.g., Bluetooth.

In one embodiment, the urine output volume monitoring device 200 further comprises an electrodermal sensor that measures the skin impedance of the patient to identify when the patient is about to urinate. This feature allows the urine output volume monitoring device 200 to scan the bladder only when the patient is about to urinate, which can eliminate the constant periodic scanning of the bladder. Alternatively, while the constant scanning of the bladder is performed, the electrodermal sensor notifies the processor 221 when the patient is about to urinate, which can enhance the accuracy of the urine output volume measurement over time.

In one embodiment, the urine output volume monitoring device 200 further comprises a flow sensor that monitors patient's fluid intake when worn on his or her neck, for example. It is known that the bodies of HF patients respond differently compared to healthy individuals such that drinking lots of water would not result in a situation where the body would simply take in the water and excrete the urine. Having the flow sensor may provide the additional information needed to make even more controlled adjustment of the diuretic dose.

As shown in FIG. 20, the diuretic administration device 300 comprises the processor 321, the injection site 310, the drug delivery tubing 312, the power source 325, the drug delivery and storage unit 330, a memory 322, a network interface 320, the control panel and indicator 332, and a speaker 323. The drug delivery and storage unit 330 includes the pumping unit 331 and the drug cartridge 336, the external views of which are shown in FIGS. 11A, 11B, and 12A-12C.

The injection site 310 is a subcutaneous injection site with an injection feature for subcutaneous diuretic injection. The drug delivery tubing 312 connects the pumping unit 331 of the drug delivery and storage unit 330 to the injection site 310. The network interface 320 is a wireless network interface controller that communicates with other external devices, such as the urine output volume monitoring device 200, according to wireless communication protocols, e.g., Bluetooth. The power source 325 is, e.g., a battery, which supplies power to the components of the diuretic administration device 300. The drug delivery and storage unit 330 stores and delivers diuretic to the patient body. The control panel and indicator 332 accepts user inputs and displays information for the user. The control panel and indicator 332 may be formed of a display screen and a control panel including one or more keys and/or a touch panel. The speaker 323 functions to alert the user with sounds when necessary and optionally to generate responsive sounds when different actions are performed by the user through the control panel and indicator 332.

Now the operation of the closed-loop home diuretic therapy system 100 in one embodiment is described with reference to FIG. 20. Initially, patient's initial diuretic dose and the target urine output volume, U_{tgt}, at the end of a specified time period, T, are determined, e.g., by a physician who possesses the proper clinical knowledge and knowledge of the patient's medical history (identified as S1 in FIG. 20), and then entered, e.g., by the physician, into the urine output volume monitoring device 200 via the control panel 217. In addition, the margin of error for the target urine output volume, ΔU_{tgt}, at the end of the specified time period, T, is entered into the urine output volume monitoring device 200 via the control panel 217. Alternatively, instead of entering the information into the urine output volume monitoring device 200, the information may be transmitted to the urine output volume monitoring device 200 from a device operated by the physician via a network. The series of steps depicted on the patient side in FIG. 20 begins when the initial diuretic dose is administered to the patient by means of the diuretic administration device 300.

The ultrasound sensor 211 of the urine output volume monitoring device 200 scans the bladder (S2) and the data are stored in the memory 222 (S3). The accelerometer 220 of the urine output volume monitoring device 200 detects the orientation of the urine output volume monitoring device 200 and stores the data in the memory 222 (S4). The bladder scan data and the orientation data are used by the processor 221 to estimate the urine inside the bladder (S5). The urine output volume over a specified time period is then determined by the processor 221 (S6). The calculated urine output volume is compared against the target urine output volume (S7) to determine whether or not the diuretic dose must be adjusted. If the diuretic dose needs to be adjusted, one of the algorithms described below is applied to determine a new diuretic dose. If the diuretic dose does not need to be adjusted, then the dose determined in step S9 would equal the most recently administered diuretic dose. Once the diuretic dose is determined (S8), it is sent to the diuretic administration device 300 (S9, S10).

Upon receiving the diuretic dose data (S11), the processor 321 of the diuretic administration device 300 executes the diuretic administration by means of the drug delivery and storage unit 330 delivering the diuretic to the subcutaneous injection site 310 through the drug delivery tubing 312 (S12). The diuretic dose data are stored in the memory 322 and sent back to the urine output volume monitoring device 200 (S13). The process described above, S2-S13, is repeated until the specified time period, T, is reached.

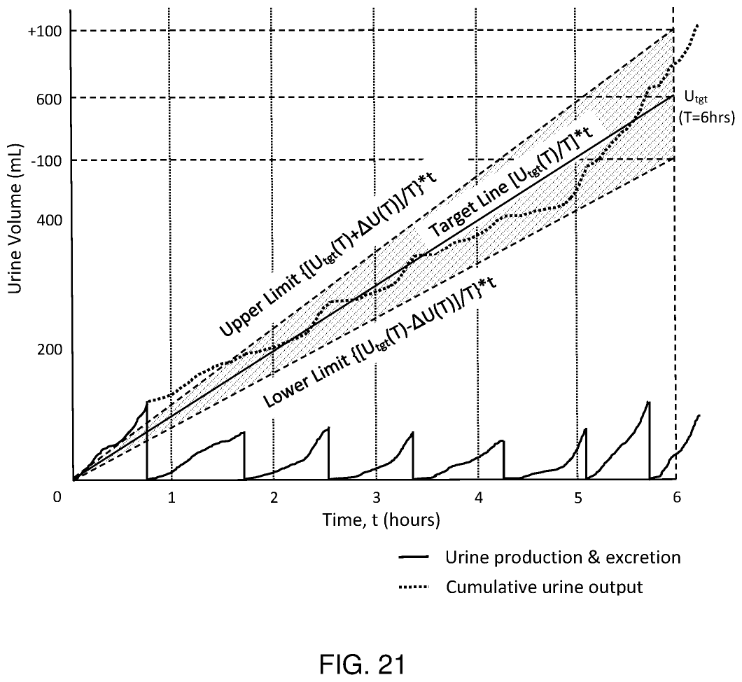

FIG. 21 is a plot of urine volume over time. As mentioned, the initial diuretic dose and the target urine output volume at the end of the specified time period are determined by a physician. Commonly used timeframe to gauge the urine output volume by a physician may be six hours. For example, a physician may target 600 mL+/−100 mL of urine output volume over six hours for a patient who is given a set diuretic dose where the target urine output volume over six hours is 600 mL and the margin of error being +/−100 mL. This would mean that the expected urine output volume over twelve hours would be 1200 mL+/−200 mL. In FIG. 21, the target urine output volume is set at 600 mL+/−100 mL.

Assuming a consistent urine volume output over time for the patient, the relationship between time and the urine output volume may be described as [U_{tgt}(T)/T]*t which is a linear curve from (0,0) to (T, U_{tgt}(T)) where the x-axis is time and the y-axis is urine output volume. This line represents the target urine output volume as a function of time, U_{tgt}(t).

ΔU_{tgt}(T) is used to determine the margin of error curves for the target urine output volume as a function of time, in which the upper limit is a linear curve from (0,0) to {[U_{tgt}(T)+ΔU_{tgt}(T)]/T}*t and the lower limit is a linear curve from (0,0) to {[U_{tgt}(T)−ΔU_{tgt}(T)]/T}*t as shown in FIG. 21. The algorithm for determining the new diuretic dose in S9 keeps the urine output volume of the patient along the target line bracketed by the margin of error curves. For example, the new diuretic dose may be determined based on a relationship between the diuretic dose and the urine output volume wherein the relationship may be represented by a sigmoid curve. According to this algorithm, when the urine output volume at time, t, is compared against U_{tgt}(t), and if it is determined that the urine output volume has deviated away from U_{tgt}(t), then the diuretic dose is adjusted in positive or negative directions accordingly.

In one embodiment, the processor 221 executes an on-off control algorithm to calculate the new diuretic dose. When the urine output volume at time, t, is compared against U_{tgt}(t), and if the urine output volume is under U_{tgt}(t)−ΔU_{tgt}(t), then the diuretic dose is increased, and if the urine outptut volume is over U_{tgt}(t)+ΔU_{tgt}(t), then the diuretic dose is decreased. This on-off control algorithm is described later.

In another embodiment, the processor 221 executes a propoertional-integral-derivative (PID) control algorithm to calculate the new diuretic dose. The coefficients for the proportional, integral, and derivative terms (i.e., Kp, Ki, and Kd) of the PID control algorithm are determined based on clinical evidence. To determine Kp from clincal evidence, for example, the initial Kp can be set as the diuretic input divided by the urine output. Ki and Kd may be introduced and all three coefficients may be adjusted according to simulated data and clinical evidence to achieve desired system response and stability. Specifically, the initial diuretic dose is determined by a physician and input to the PID controller, and the new diuretic dose is determined according to the monitored urine output volume so that it reaches and does not deviate from the target urine output volume. This Kp and other coefficients Ki and Kd may be adjusted according to clinical evidence as necessary.

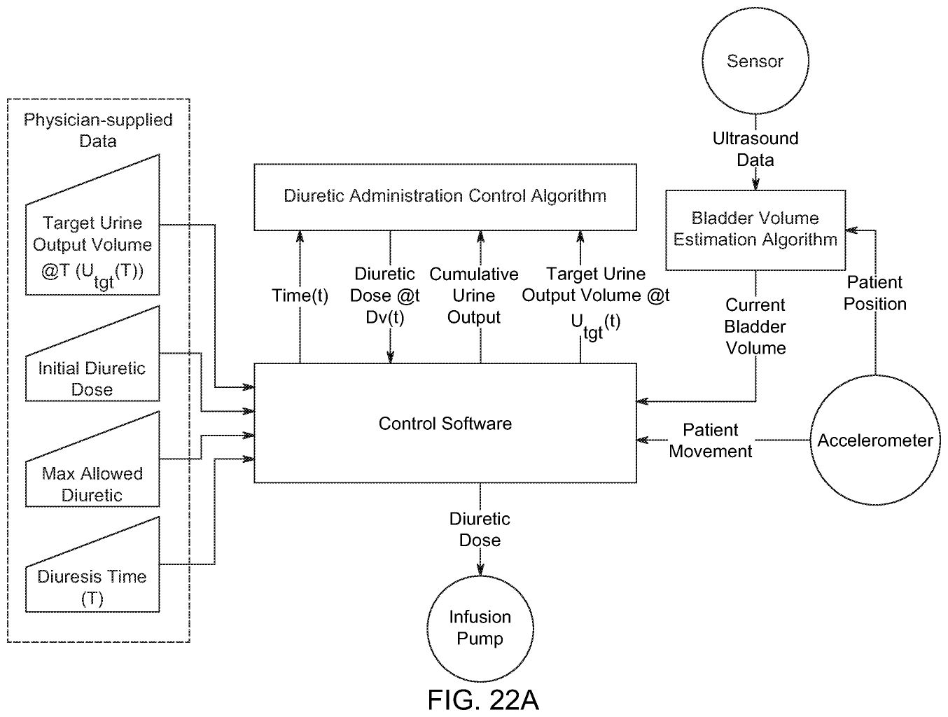

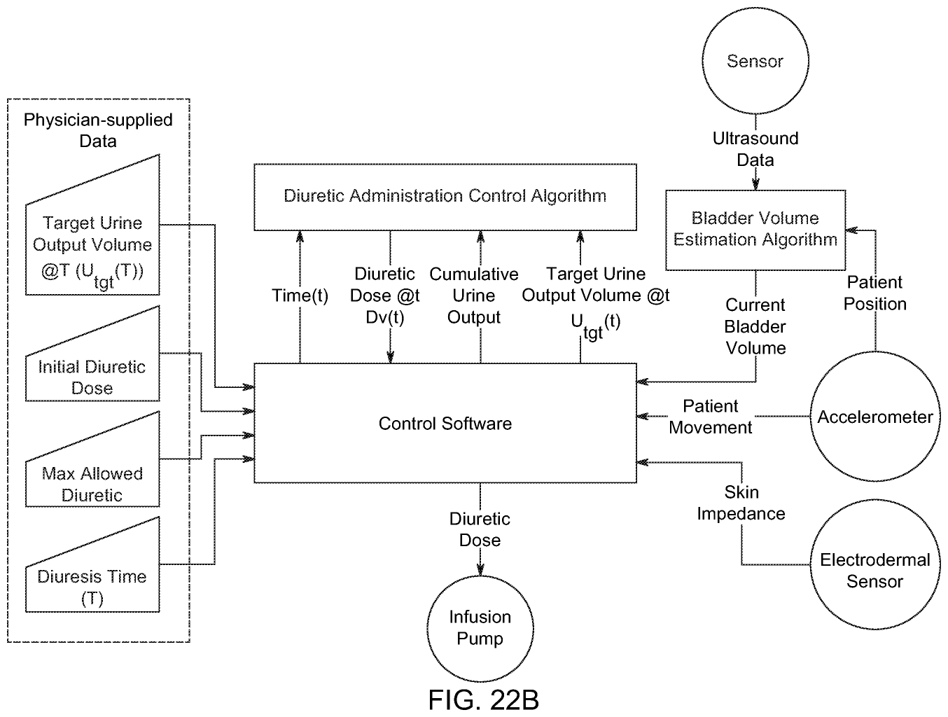

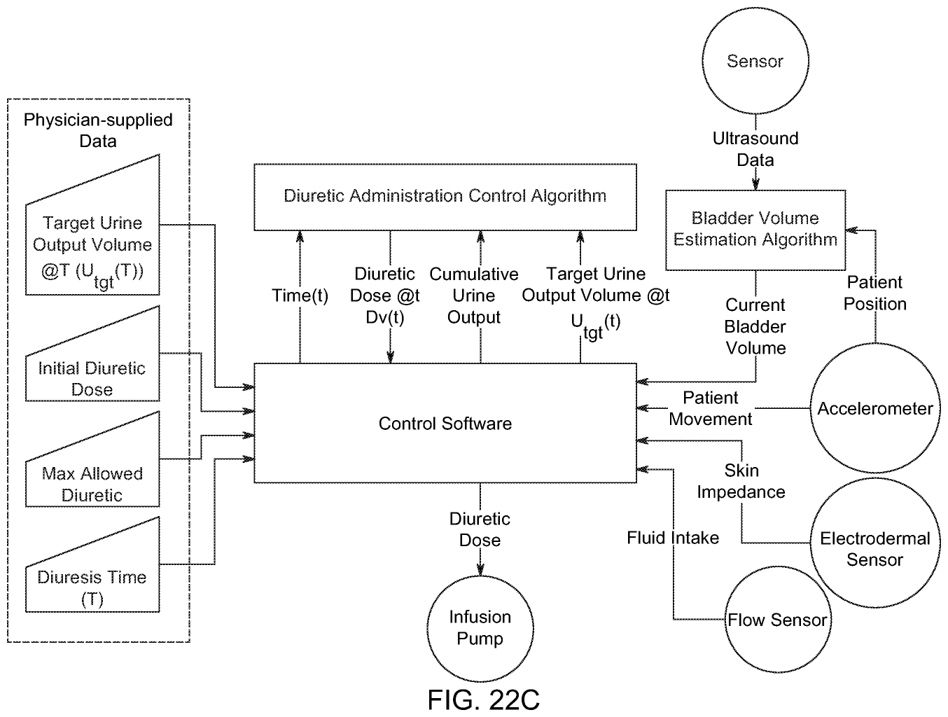

FIGS. 22A, 22B, and 22C depict the flow of data in the closed-loop home diuretic therapy system 100 in accordance with an exemplary embodiment. As shown in these figures, four parameters determined by a physician, i.e., the target urine output volume U_{tgt}(T), the initial diuretic dose, the maximum allowed diuretic dose, and the diuresis time T, are stored in the urine output volume monitoring device 200 and processed by control software installed therein. Once the therapy has been started, one or more sensors (e.g., the ultrasound sensor 211, the accelerometer 220, the electrodermal sensor, and/or the flow sensor) collect and input data to the control software. In particular, the data from the ultrasound sensor 211 and the accelerometer 220 are used to estimate the current bladder volume of the patient, which is used to determine the cumulative urine output. The control software uses the diuretic administration control algorithm to calculate the new diuretic dose as necessary and the calculated diuretic dose is administered to the patient via the diuretic administration device 300.

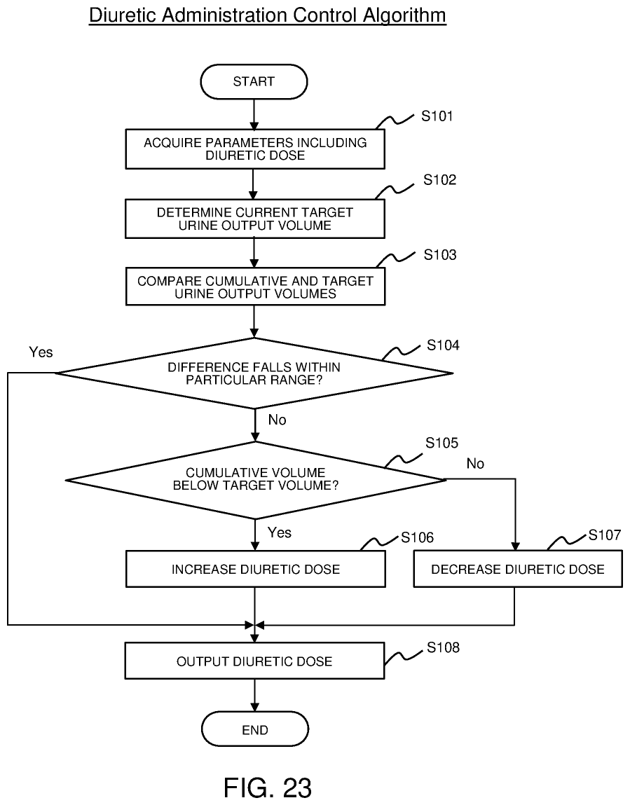

FIG. 23 is a flowchart of steps performed by the closed-loop home diuretic therapy system 100 according to the diuretic administration on-off control algorithm. In this example, the steps are repeatedly performed by the processor 221 of the urine output volume monitoring device 200 at a predetermined time interval. However, they may be performed by the processor 321 of the diuretic administration device 300 or a processor of the mobile device 500.

Initially, the processor 221 acquires parameters including an initial diuretic dose and a target urine output volume after a specified time period, which are stored in the memory 222 (S101). Next, the processor 221 determines a current target urine output volume based on the acquired target urine output volume and sensor outputs (S102). Subsequently, the processor 221 compares the determined current target urine output volume with the cumulative urine output volume (S103). Here, the cumulative urine output volume can be estimated from changes in the bladder volume according to known methods.

Based on the comparison result, the processor determines whether a difference between the determined current target urine output volume and the cumulative urine output volume falls within a particular range (S104). If so (Yes, S104), the processor 221 outputs the diuretic dose same as the one acquired at S101 (S108). If not (No, S104), the processor 221 determines whether the cumulative urine output volume is below the current target urine output volume (S105). When the cumulative urine output volume is below the determined current target urine output volume (Yes, S105), the processor 221 increases the diuretic dose by a particular amount and outputs the increased diuretic dose (S108). On the other hand, when the cumulative urine output volume exceeds the determined current target urine output volume (No, S105), the processor 221 decreases the diuretic dose by the particular amount and outputs the decreased diuretic dose (S108). The output diuretic dose is stored in the memory 222 and transmitted to the diuretic administration device 300.

In the example of FIG. 23, the diuretic dose is adjusted depending on whether the urine output falls outside the target window according to the on-off control algorithm. However, such adjustment can be made according to a known PID control as described above. For example, the processor 221 may base the difference between the current cumulative and target urine output volumes, the integrated value of the past differences, and the rate of change in such differences to determine the new diuretic dose.

The detailed description above describes embodiments of a closed-loop home diuretic therapy system, and more particularly, a closed-loop system and methods for achieving home diuretic therapy for people with heart failure (HF) with a urine output volume monitoring device, a diuretic administration device, a closed-loop algorithm to regulate diuretic dose based on the urine output volume, and a mobile device app to provide operational and informational interface to the user, which are configured, for example, to achieve fluid balance of people who suffer from fluid volume overload caused by HF. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

One or more embodiments are specified in the following paragraphs:

1. A closed-loop home diuretic therapy system configured to achieve decongestion without residual volume overload in a patient, the therapy system comprising: a urine output volume monitoring device to be attached to the patient, the urine output volume monitoring device having a sensor to detect a wall of a bladder of the patient, an accelerometer to detect an orientation of urine output volume monitoring device, and a processor to determine an amount of urine in the bladder based on the detected wall and calculate urine output volume based on the detected orientation and the determined amount of urine in the bladder; and a diuretic administration device configured to communicate with the urine output volume monitoring device, the diuretic administration device having a subcutaneous injection site with an injection feature for subcutaneous diuretic injection, a diuretic delivery and storage device with a pump to deliver diuretic stored therein to the subcutaneous injection site, and a control circuit having a processor that is configured to regulate the volume and rate of diuretic delivered from the diuretic delivery and storage device to the subcutaneous injection site based on the calculated urine output volume and a target volume and rate of diuretic to be administered.
2. The closed-loop home diuretic therapy system according to paragraph 1, wherein the sensor is an ultrasound sensor.
3. The closed-loop home diuretic therapy system according to paragraph 2, wherein the ultrasound sensor includes a plurality of sensor elements arranged in an array to cover the entire profile of the bladder in all anatomical directions.
4. The closed-loop home diuretic therapy system according to paragraph 2, wherein the sensor elements are configured to change the orientation of the ultrasound sensor or the direction of ultrasound signals to cover the entire profile of the bladder in all anatomical directions.
5. The closed-loop home diuretic therapy system according to paragraph 2, wherein the ultrasound sensor is incorporated in a wearable component, which includes a medium to reduce or eliminate an air gap between the urine output volume device and a contact area on the patient to enhance passing of ultrasound signals through human tissue.
6. The closed-loop home diuretic therapy system according to paragraph 5, wherein the medium contains fluid, absorbs fluid, or allows fluid to pass through, and is replaceable.
7. The closed-loop home diuretic therapy system according to paragraph 1, wherein the urine output volume monitoring device utilizes an acoustic sensor to detect a rate of urine flow through a urethra of the patient and the processor of the urine output volume monitoring device calculates the urine output volume further based on the detected rate of urine flow.
8. The closed-loop home diuretic therapy system according to paragraph 7, wherein the urine output volume monitoring device utilizing the acoustic sensor is placed on a perineum of the patient.
9. The closed-loop home diuretic therapy system according to paragraph 1, wherein the urine output volume monitoring device utilizes an adhesive to be attached to a skin of the patient.
10. The closed-loop home diuretic therapy system according to paragraph 1, wherein the urine output volume monitoring device utilizes an adjustable belt strap to be worn by the patient.
11. The closed-loop home diuretic therapy system according to paragraph 1, wherein the urine output volume monitoring device utilizes an undergarment-type configuration to be worn by the patient.
12. The closed-loop home diuretic therapy system according to paragraph 1, wherein the diuretic administration device utilizes diuretic that is formulated for subcutaneous injection.
13. The closed-loop home diuretic therapy system according to paragraph 1, wherein the diuretic administration device utilizes an array of microneedles to be placed on a skin of the patient for subcutaneous injection.
14. The closed-loop home diuretic therapy system according to paragraph 1, wherein the diuretic administration device utilizes a cannula to be placed under a skin of the patient for subcutaneous injection.
15. The closed-loop home diuretic therapy system according to paragraph 1, wherein the diuretic administration device utilizes an adhesive for the injection site to be attached to a skin of the patient.
16. The closed-loop home diuretic therapy system according to paragraph 1, wherein the diuretic administration device utilizes an adjustable belt strap for the injection site to be worn by the patient.
17. The closed-loop home diuretic therapy system according to paragraph 1, wherein the diuretic administration device utilizes a clip-on attachment to place the diuretic delivery and storage device on the patient.
18. The closed-loop home diuretic therapy system according to paragraph 1, wherein the amount of urine is adjusted based on generally known anatomical bladder positions for different body positions.
19. The closed-loop home diuretic therapy system according to paragraph 1, wherein the processor of the urine output volume monitoring device is configured to trigger an alarm to alert the patient to be in a stable body position.
20. The closed-loop home diuretic therapy system according to paragraph 1, wherein the processor of the diuretic administration device calculates the volume and rate of diuretic to be delivered from the diuretic delivery and storage device to the subcutaneous injection site based on the calculated urine output volume and the target volume and rate of diuretic to be administered.
21. The closed-loop home diuretic therapy system according to paragraph 20, wherein the diuretic administration device is configured to generate an alert if the calculated rate of diuretic to be delivered from the diuretic delivery and storage device to the subcutaneous injection site is not in a specified range.
22. The closed-loop home diuretic therapy system according to paragraph 1, further comprising: an application running on a mobile device to calculate the volume and rate of diuretic to be delivered from the diuretic delivery and storage device to the subcutaneous injection site based on the calculated urine output volume and the target volume and rate of diuretic to be administered, and to display to the patient the information on the urine output volume, the diuretic administration amount and rate, and status based on a target urine output volume and an administered diuretic volume.
23. The closed-loop home diuretic therapy system according to paragraph 22, wherein the application running on the mobile device is configured to generate an alert if the calculated rate of diuretic to be delivered from the diuretic delivery and storage device to the subcutaneous injection site is not in a specified range.
24. A method of achieving diuretic therapy at home, the method comprising: (a) monitoring a urine output volume of a person by a wearable or implantable device, detecting an orientation of the wearable or implantable device, and calculating the urine output volume using the detected orientation; (b) determining the amount and rate of diuretic to be administered utilizing the calculated urine output volume and a medical history of the person; (c) administering diuretic to the person by subcutaneous injection in accordance with the determined amount and rate of the diuretic; and repeating steps (a), (b), and (c) until a calculated urine output volume corresponding to administered diuretic volume and rate matches a target urine output volume.
25. The method according to paragraph 24, wherein the amount and rate of diuretic to be administered is determined based on the person's electrolyte level, blood pressure, weight, or any combination of the above.
26. The method according to paragraph 24, further comprising: sending the calculated urine output volume to a cloud computing system accessible by healthcare professionals, and receiving feedback of the healthcare professionals on the diuretic amount and rate to be administered from the cloud computing system.
27. The method according to paragraph 24, further comprising: sending the calculated and stored information and data from the urine output volume to a cloud computing system accessible by healthcare professionals, who utilize the person's electrolyte level, blood pressure, weight, any or combination of the above to prepare feedback on the diuretic amount and rate to be administered, and receiving the feedback of the healthcare professionals on the diuretic amount and rate to be administered from the cloud computing system.
28. A diuretic therapy system to be worn on a body of a patient, the diuretic therapy system comprising: a urine output volume monitoring device to be worn on the body of the patient, the urine output volume monitoring device having a sensor to detect a wall of a bladder of the patient, an accelerometer to detect the an orientation of the urine output volume monitoring device to account for different bladder positions of the patient, and a processor to determine an amount of urine in the bladder based on the detected wall and calculate urine output volume based on the detected orientation and the determined amount of urine in the bladder; and a diuretic administration device configured to communicate with the urine output volume monitoring device and to be worn on the body of the patient, the diuretic administration device having a subcutaneous injection site with an injection feature for subcutaneous diuretic injection, a diuretic delivery and storage device with a pump to deliver diuretic stored therein to the subcutaneous injection site, and a control circuit having a processor that is configured to regulate the volume and rate of diuretic delivered from the diuretic delivery and storage device to the subcutaneous injection site based on the calculated urine output volume and target volume and rate of diuretic to be administered.
29. A closed-loop home diuretic therapy system configured to achieve decongestion without residual volume overload in a patient, the therapy system comprising: a urine output volume monitoring device to be attached to the patient, the urine output volume monitoring device having a sensor to detect a wall of a bladder of the patient, an accelerometer and/or a gyroscope to detect acceleration of the urine output volume monitoring device, and a processor to determine an amount of urine in the bladder based on the detected wall and the detected acceleration and calculate urine output volume using the determined amount of urine; and a diuretic administration device configured to communicate with the urine output volume monitoring device, the diuretic administration device having a subcutaneous injection site with an injection feature for subcutaneous diuretic injection, a diuretic delivery and storage device with a pump to deliver diuretic stored therein to the subcutaneous injection site, and a control circuit having a processor that is configured to regulate the volume and rate of diuretic delivered from the diuretic delivery and storage device to the subcutaneous injection site based on the calculated urine output volume and a target volume and rate of diuretic to be administered.
30. The closed-loop home diuretic therapy system according to paragraph 29, wherein the processor of the urine output volume monitoring device determines the amount of urine in the bladder by: determining a volume of the bladder based on the detected wall and/or the detected acceleration, determining an error of the determined volume using the detected acceleration, and then determining the amount of urine in the bladder based on the determined volume and error.
31. A closed-loop diuretic therapy system, comprising: a drug delivery and storage unit configured to store diuretic and deliver the diuretic into a body of a patient via a subcutaneous injection site; a sensor configured to detect a bladder wall of the patient; and a processor configured to: store in a memory a diuretic dose and a target urine output volume over a particular time period, monitor a change in a volume of the bladder based on an output from the sensor and determine a cumulative urine output volume based on the change, and modify the diuretic dose stored in the memory based on a deviation of the cumulative urine output volume from the target urine output volume, wherein the drug delivery and storage unit delivers the diuretic according to the diuretic dose stored in the memory.
32. The closed loop diuretic therapy system according to paragraph 31, wherein the processor is further configured to: determine a current target urine output volume at a current time based on the stored target urine output volume and the time period, calculate a difference between the cumulative urine output volume and the current target urine output volume, and modify the diuretic dose based on the calculated difference.
33. The closed loop diuretic therapy system according to paragraph 31, further comprising: an electrodermal sensor configured to measure an impedance of a skin of the patient, wherein the processor is further configured to determine when the sensor detects the bladder wall based on the measured impedance of the skin.
34. The closed loop diuretic therapy system according to paragraph 31, further comprising: a flow sensor configured to monitor fluid intake by the patient, wherein the processor modifies the diuretic dose further based on the monitored fluid intake.
35. The closed loop diuretic therapy system according to paragraph 31, further comprising: a housing in which the sensor and the processor are disposed, and an attachment connected to the housing and having an adhesive surface to be attached to the body of the patient.
36. The closed loop diuretic therapy system according to paragraph 35, further comprising: a holder connected to the attachment and by which the housing is held.
37. The closed loop diuretic therapy system according to paragraph 35, wherein the sensor includes a plurality of sensor elements that are arranged along the adhesive surface.
38. The closed loop diuretic therapy system according to paragraph 37, wherein the sensor elements are arranged in a plurality of rows such that a different number of sensor elements are arranged in each of two of the plurality of rows that are adjacent to each other.
39. The closed loop diuretic therapy system according to paragraph 37, wherein the sensor elements are arranged in a circular or oval pattern.
40. The closed loop diuretic therapy system according to paragraph 37, wherein each of the sensor elements is configured to emit ultrasound signals towards different directions.
41. The closed loop diuretic therapy system according to paragraph 35, further comprising: an input device in the housing and through which the diuretic dose and the target urine output volume can be set.
42. The closed loop diuretic therapy system according to paragraph 35, further comprising: another sensor configured to detect an orientation of the housing, wherein
the processor monitors the change in the volume of the bladder further based on an output from the other sensor.
   43. The closed loop diuretic therapy system according to paragraph 31, wherein the sensor is configured to move in rocking motion or rotate when emitting ultrasound signals towards the bladder wall.
   44. The closed loop diuretic therapy system according to paragraph 31, further comprising: a housing in which the sensor and the processor are disposed, and a belt to which the housing is attachable and to be worn by the patient.
   45. The closed loop diuretic therapy system according to paragraph 31, further comprising: a housing in which the sensor and the processor are disposed, and an undergarment to which the housing is attachable and to be worn by the patient.
   46. The closed loop diuretic therapy system according to paragraph 31, wherein the drug delivery and storage unit includes: a drug cartridge in which the diuretic can be stored, and a pumping unit connectable to the drug cartridge and configured to pump the diuretic stored in the drug cartridge to the subcutaneous injection site.
   47. The closed loop diuretic therapy system according to paragraph 46 wherein the subcutaneous injection site includes an attachment on which one or more microneedles are disposed and attachable to the body of the patient, and the microneedles are connected to the pumping unit via a tube.
   48. The closed loop diuretic therapy system according to paragraph 46, wherein the drug cartridge and the pumping unit are engageable via one or more connectors.
   49. A closed-loop diuretic therapy system, comprising: a drug administration device configured to store diuretic and deliver the diuretic into a body of a patient by subcutaneous injection; and a monitoring device including: a network interface wirelessly connectable to the drug administration device, a sensor configured to detect a bladder wall of the patient, and a processor configured to: store in a memory a diuretic dose and a target urine output volume over a particular time period, monitor a change in a volume of the bladder based on an output from the sensor and determine a cumulative urine output volume based on the change, modify the diuretic dose based on a deviation of the cumulative urine output volume from the target urine output volume, and control the network interface to transmit to the drug administration device a signal that causes the drug administration device to deliver the diuretic according to the modified diuretic dose.
   50. A method of achieving diuretic therapy for a patient, the method comprising: storing in a memory a diuretic dose and a target urine output volume over a particular time period; monitoring a change in a volume of a bladder of the patient using an output from a sensor configured to detect a bladder wall of the patient and determining a cumulative urine output volume based on the change; modifying the diuretic dose based on a deviation of the cumulative urine output volume from the target urine output volume; and delivering diuretic into a body of the patient by subcutaneous injection according to the modified diuretic dose.

1. A closed-loop home diuretic therapy system configured to achieve decongestion without residual volume overload in a patient, the therapy system comprising:
a urine output volume monitoring device to be attached to the patient, the urine output volume monitoring device having a sensor to detect a wall of a bladder of the patient, an accelerometer to detect an orientation of urine output volume monitoring device, and a processor to determine an amount of urine in the bladder based on the detected wall and the detected orientation and calculate urine output volume based on the determined amount of urine in the bladder; and
a diuretic administration device configured to communicate with the urine output volume monitoring device, the diuretic administration device having a subcutaneous injection site with an injection feature for subcutaneous diuretic injection, a diuretic delivery and storage device with a pump to deliver diuretic stored therein to the subcutaneous injection site, and a control circuit having a processor that is configured to regulate the volume and rate of diuretic delivered from the diuretic delivery and storage device to the subcutaneous injection site based on the calculated urine output volume and a target volume and rate of diuretic to be administered.

2. The closed-loop home diuretic therapy system according to claim 1, wherein the sensor is an ultrasound sensor.

3. The closed-loop home diuretic therapy system according to claim 2, wherein the ultrasound sensor includes a plurality of sensor elements arranged in an array to cover the entire profile of the bladder in all anatomical directions.

4. The closed-loop home diuretic therapy system according to claim 2, wherein the sensor elements are configured to change the orientation of the ultrasound sensor or the direction of ultrasound signals to cover the entire profile of the bladder in all anatomical directions.

5. The closed-loop home diuretic therapy system according to claim 2, wherein the ultrasound sensor is incorporated in a wearable component, which includes a medium to reduce or eliminate an air gap between the urine output volume device and a contact area on the patient to enhance passing of ultrasound signals through human tissue.

6. The closed-loop home diuretic therapy system according to claim 5, wherein the medium contains fluid, absorbs fluid, or allows fluid to pass through, and is replaceable.

7. The closed-loop home diuretic therapy system according to claim 1, wherein the urine output volume monitoring device utilizes an acoustic sensor to detect a rate of urine flow through a urethra of the patient and the processor of the urine output volume monitoring device calculates the urine output volume further based on the detected rate of urine flow.

8. The closed-loop home diuretic therapy system according to claim 7, wherein the urine output volume monitoring device utilizing the acoustic sensor is placed on a perineum of the patient.

9.-11. (canceled)

12. The closed-loop home diuretic therapy system according to claim 1, wherein the diuretic administration device utilizes diuretic that is formulated for subcutaneous injection.

13.-17. (canceled)

18. The closed-loop home diuretic therapy system according to claim 1, wherein the amount of urine is adjusted based on generally known anatomical bladder positions for different body positions.

19. The closed-loop home diuretic therapy system according to claim 1, wherein the processor of the urine output volume monitoring device is configured to trigger an alarm to alert the patient to be in a stable body position.

20. The closed-loop home diuretic therapy system according to claim 1, wherein
the processor of the diuretic administration device calculates the volume and rate of diuretic to be delivered from the diuretic delivery and storage device to the subcutaneous injection site based on the calculated urine output volume and the target volume and rate of diuretic to be administered.

21. The closed-loop home diuretic therapy system according to claim 20, wherein
the diuretic administration device is configured to generate an alert if the calculated rate of diuretic to be delivered from the diuretic delivery and storage device to the subcutaneous injection site is not in a specified range.

22. The closed-loop home diuretic therapy system according to claim 1, further comprising:
an application running on a mobile device to calculate the volume and rate of diuretic to be delivered from the diuretic delivery and storage device to the subcutaneous injection site based on the calculated urine output volume and the target volume and rate of diuretic to be administered, and to display to the patient the information on the urine output volume, the diuretic administration amount and rate, and status based on a target urine output volume and an administered diuretic volume.

23. The closed-loop home diuretic therapy system according to claim 22, wherein
the application running on the mobile device is configured to generate an alert if the calculated rate of diuretic to be delivered from the diuretic delivery and storage device to the subcutaneous injection site is not in a specified range.

24. A method of achieving diuretic therapy at home, the method comprising:
(a) monitoring a urine output volume of a person by a wearable or implantable device, detecting an orientation of the wearable or implantable device, and calculating the urine output volume using the detected orientation;
(b) determining the amount and rate of diuretic to be administered utilizing the calculated urine output volume and a medical history of the person;
(c) administering diuretic to the person by subcutaneous injection in accordance with the determined amount and rate of the diuretic; and
repeating steps (a), (b), and (c) until a calculated urine output volume corresponding to administered diuretic volume and rate matches a target urine output volume.

25. The method according to claim 24, wherein
the amount and rate of diuretic to be administered is determined based on the person's electrolyte level, blood pressure, weight, or any combination of the above.

26. The method according to claim 24, further comprising:
sending the calculated urine output volume to a cloud computing system accessible by healthcare professionals, and receiving feedback of the healthcare professionals on the diuretic amount and rate to be administered from the cloud computing system.

27. The method according to claim 24, further comprising:
sending the calculated and stored information and data from the urine output volume to a cloud computing system accessible by healthcare professionals, who utilize the person's electrolyte level, blood pressure, weight, any or combination of the above to prepare feedback on the diuretic amount and rate to be administered, and receiving the feedback of the healthcare professionals on the diuretic amount and rate to be administered from the cloud computing system.

28. A diuretic therapy system to be worn on a body of a patient, the diuretic therapy system comprising:
a urine output volume monitoring device to be worn on the body of the patient, the urine output volume monitoring device having a sensor to detect a wall of a bladder of the patient, an accelerometer to detect the an orientation of the urine output volume monitoring device to account for different bladder positions of the patient, and a processor to determine an amount of urine in the bladder based on the detected wall and the detected orientation and calculate urine output volume based on the determined amount of urine in the bladder; and
a diuretic administration device configured to communicate with the urine output volume monitoring device and to be worn on the body of the patient, the diuretic administration device having a subcutaneous injection site with an injection feature for subcutaneous diuretic injection, a diuretic delivery and storage device with a pump to deliver diuretic stored therein to the subcutaneous injection site, and a control circuit having a processor that is configured to regulate the volume and rate of diuretic delivered from the diuretic delivery and storage device to the subcutaneous injection site based on the calculated urine output volume and target volume and rate of diuretic to be administered.
